(19) United States
(12) Patent Application Publication
(10) Pub. No.: US 2023/0295570 A1
(43) Pub. Date: Sep. 21, 2023

(54) CLOSED MANUFACTURING PROCESSES FOR LARGE SCALE MANUFACTURING OF PLURIPOTENT STEM CELL DERIVED CELLS

(71) Applicant: NCARDIA B.V., Leiden (NL)

(72) Inventor: Stefan Robbert BRAAM, Leiden (NL)

(21) Appl. No.: 18/017,107

(22) PCT Filed: Jul. 23, 2021
(86) PCT No.: PCT/NL2021/050474

§ 371 Date: Jan. 20, 2023

(30) Foreign Application Priority Data
Jul. 23, 2020 (NL) 2026121

(51) Int. Cl. C12N 5/0789; C12N 5/071; C12N 5/0793; C12M 1/00

(52) U.S. Cl. CPC C12N 5/0647; C12N 5/069; C12N 5/0619; C12M 23/14; C12M 23/58; C12N 2501/727; C12N 2513/00; C12N 2500/35; C12N 2506/45; C12N 2501/165; C12N 2501/125; C12N 2501/115; C12N 2501/155

(57) ABSTRACT

The present invention is in the field of pluripotent stem cells. In particular the invention relates to a method for (closed system) induction of differentiation of pluripotent stem cells towards a pre-selected cell type, such as, for example, cardiomyocytes or endothelial cells. The method as disclosed herein is particularly useful to upscale the production of cells derived from pluripotent stem cells, in particular (human) cardiomyocytes and/or endothelial cells derived from pluripotent stem cells.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Therapies based on the application of stem cells are considered promising throughout the medical field. Especially the availability of pluripotent stem cells (PSCs) with their potential for proliferation and differentiation is considered a promising development for cellular therapies (also referred to as cell therapy, cell replacement therapy or cell-based therapy) in the clinic. Pluripotent stem cells, such as induced pluripotent stem cells and embryonic pluripotent stem cells, are able, due to their pluripotency, to differentiate into target cells for the intended therapeutic use. The availability of, for example, neuronal cells, retinal cells, lung cells, liver cells, pancreatic cells, cardiovascular cells, or cells of the immune system, obtained ex vivo from such pluripotent stem cells, for cellular therapy would be most welcome.

One example of a potential therapeutic use for such pluripotent stem cell-derived cells is the replacement of irreversibly damaged myocardium, using cardiomyocytes, endothelial, fibroblast and or any combination of these cells to treat myocardial infarction (see, for example, Cell & Gene Therapy Insights 2020; 6(1), 177-191 DOI: 10.18609/cgti.2020.023).

Another example of therapeutic uses for such pluripotent stem cell-derived cells can be the treatment of cancer using allogenic or autologous immune cells carrying antigen receptors targeted against the tumor. A further example may be the use of (induced) pluripotent stem cell-derived lymphocytes for adoptive cell immunotherapy (see, for example, Curr Hematol Malig Rep. 2019; 14(4): 261-268 doi: 10.1007/s11899-019-00528-6).

The potential therapeutic uses, such as the one exemplified here above, requires large numbers of pluripotent stem cell-derived cells. In a myocardial infarction for example, it may be that over a billion cardiomyocytes are damaged irreversibly. With current protocols available this still requires an enormous investment in time and materials to be able to manufacture sufficient amounts of cells under clinical Good Manufacturing Practice (cGMP) conditions.

For other indications, such as disease of the eye, smaller amounts of cells are sufficient to treat one patient, but manufacturing of enough cells for the global patient population still requires scaling of manufacturing processes in order to reach the required capacity.

A major drawback is that methods known in the art for differentiating of (induced) iPSC to a preselected, desired, cell type or cell types (e.g. a liver cell) rely on labor intensive procedures in small dishes or flasks yielding maximal a few million cells per dish, which makes manufacturing sufficient cells for treatment the number one challenge in the field of cell therapy.

Therefore, to fulfil demand of industrially applicable scalable manufacturing procedures it is required that the field provides scalable methods and that can be compatible with clinical Good Manufacturing Practice (cGMP). Only with the availability of such methods it will become possible to produce vast amounts of pluripotent stem cells, and, more importantly, vast amounts of (preselected) pluripotent stem cell-derived differentiated cells, including those mentioned above. Upscaling the present culturing, differentiation and manufacturing processes in a manner that allows safe, non-disturbed, controllable, predictable, less handling intense and less labor intense production of pluripotent stem cell derived differentiated cells is therefore highly desirable.

It is well established in the art that differentiation of pluripotent stem cells towards cells of different lineages can be induced and controlled by exposing pluripotent stem cells to particular culture conditions or regimens using culture media comprising specific (combinations of) small molecules and other steering compounds (see, for example, Breckwoldt et al. Nat Protoc. 2017 June; 12(6):1177-1197. doi: 10.1038/nprot.2017.033 or Induced Pluripotent Stem Cells—Methods and Protocols (Turksen and Nagy); doi: 10.1007/978-1-4939-3055-5). The (combinations of) small molecules or steering compounds are included to, for example, agonize or antagonize particular pathways that steer differentiation during a particular stage of the differentiation of the cells. This means that it is considered important in the field to modulate the right pathway at the right time during differentiation. Similarly, it is considered important that pathways that antagonize or counteract pathways that are desired during a particular stage of differentiation are not activated or are to be antagonized. Indeed, it is not uncommon that, for example, a pathway that needs to be antagonized during a first stage of the differentiation in order to differentiate towards a pre-selected cell type does not play a role during later stages of differentiation and should no longer be antagonized, or should even be agonized during such later stage of differentiation as it may negatively influence differentiation during a later stage of the differentiation (see for example, European patent document EP3433355).

Methods for differentiation of cells on a small scale are performed by manipulating the cells in a biosafety cabinet and culturing in an incubator, and medium replacement with a further medium (for example replacement of a first medium comprising a first, e.g. agonistic, steering compound by a second medium comprising a second, e.g. antagonistic, steering compound for the same pathway) are performed by means of manual handling. However, the field is looking for culturing systems wherein manual handling is reduced to a minimum. Manual handling, namely, is difficult to scale, very expensive and brings significant risks of contamination and breach of the sterility of the culture.

At the same time, there is a large need to improve reproducibility and consistency of manufacturing. Real time adjustments of process parameters in manual culture systems is labor intensive and therefore difficult to implement. Thus, the field is seeking manufacturing systems that may be monitored and where adjustments can be made where appropriate.

At the same time, the field is looking to expand the scale of manufacturing of differentiated cells obtained from pluripotent stem cells. Scale out strategies based on multiple flasks is however extremely labor intensive and time-consuming. In order to provide large numbers of cells hundreds of flasks are required, carrying significant challenges with harvesting and down-stream processing the cells as a single batch. In addition there is the risk of significant differences in the quality of the cells per flask (see e.g. Assou et al (2018) Stem Cells 36, 814-821).

Multiple initiatives in the fields have therefore been initiated to provide new approaches enabling scale-up of pluripotent stem cell culturing. However, the limitations of such systems are that the systems still require, during cultivation, centrifugation steps, filtration steps and/or wash steps, for example, before proceeding to the next stage of the differentiation of the cells, such as for example described in WO2009/072003.

Methods for culturing pluripotent stem cells in bioreactors, described in the art, are mainly directed to maintaining/sustaining a stem cell population in culture. In such methods the culture is maintained by means of continuous stirring, wherein once so often, the medium wherein cells are cultured is manually replaced, thereby risking a breach in the sterility of the system. Other previous described methods describing stirred tank bioreactors for culturing pluripotent stem cells utilize a perfusion system for medium exchange in the closed system. Disadvantages of such perfusion systems are the risk of filter blockage especially if larger amounts of medium have to be exchanged during the culturing.

Importantly, scale-up of pluripotent stem cell derived cell manufacturing typically requires providing a series of different media to the pluripotent stem cells and/or pluripotent stem cell-derived cells. In such process towards obtaining differentiated cells the series of media comprise combinations of (different) compounds that prepare and/or induce cells towards the directed differentiation. However, methods presently known in the art utilizing closed system cultivation are limited to maintaining or producing pluripotent stem cells and no reliable, predictable, easy-to-handle cultivation methods allowing the production of vast amounts of pluripotent stem cells-derived differentiated cells in a controllable fashion and in a closed culture system are currently available and this bottleneck in the application of cell therapy is widely recognized in the field.

In light of this, new methods in the cultivation and production of vast amounts of (preselected) stem-cell derived differentiated cells are highly desirable. In particular, there is a clear need in the art for reliable, efficient and reproducible methods and that allow manufacturing of vast amount of different types of pluripotent stem cell derived differentiated cells.

Accordingly, the technical problem underlying the present invention can been seen in the provision of such methods for complying with any of the aforementioned needs. The technical problem is solved by the embodiments characterized in the claims and herein below.

### DESCRIPTION

### Drawings

Embodiments of the invention are further described hereinafter with reference to the accompanying drawings, in which:

FIG. 1: Example of schematic manufacturing setup according to the invention. Closed system for manufacturing of iPSC differentiated cells, involving medium storage bag at 4° C., break tank at 37° C., and a collection bag. Pumps are connected to tubing to pump medium in and out of the bioreactor. NaHCO3 may be separately supplied for pH control. The bioreactor may have a pH probe for online corrections of pH (not shown). Medium bags can be connected to the system using sterile welding.

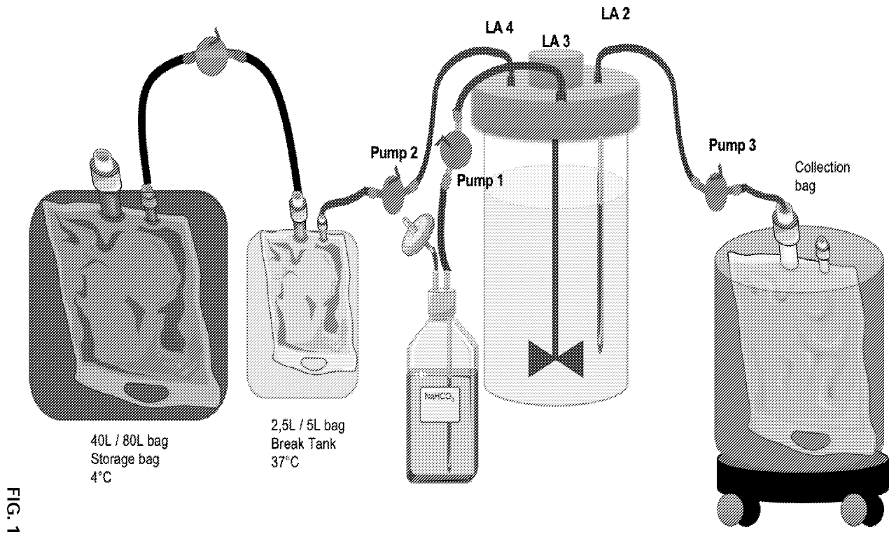

### DEFINITIONS

A portion of this disclosure contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a”, “an”, and “the” include plural referents unless the context clearly dictates otherwise. Thus, for example, reference to “a cell” includes a combination of two or more cells, and the like.

As used herein the term “about” and “approximately”, when referring to a measurable value such as an amount, a temporal duration and the like, is meant to encompass variations of ±20%, ±10% more preferably ±5%, even more preferably ±1%, still more preferably ±0.1% from said measurable value, in such way the variations are appropriate to perform the disclosed methods.

As used herein, the term “and/or” refers to a situation wherein one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, etc. As used herein, the term “at most” a particular value means that particular value or less. For example, “at most 5” is understood to be the same as “5 or less” i.e., 5, 4, 3, . . . −10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, “conventional techniques” or “methods known to the skilled person” refer to a situation wherein the methods of carrying out the conventional techniques used in methods as disclosed herein will be evident to the skilled worker. The practice of conventional techniques in molecular biology, biochemistry, cell culture, genomics, sequencing, medical treatment, pharmacology, immunology and related fields are well-known to those of skill in the art and are discussed, in various handbooks and literature references.

As used herein, “exemplary” means “serving as an example, instance, or illustration,” and should not be construed as excluding other configurations disclosed herein.

As used herein, “aggregate”, “aggregation” and “aggregated” in connection to cells refer to one of several main types of cell organization, namely the joining or clustering of a cell with another cell, or cells. Moreover, it does not comprise the joining of a cell with a substrate, commonly referred to as “adherence”. Aggregation of cells is based on cell-cell interactions. Such interactions can be formed between cells through cell surface proteins and are normally present in many biological systems such as tissues, organs and the like. Aggregation of cells can be induced or maintained in vitro by stirring or mixing a culture medium comprising (pluripotent stem) cells. When stirring or mixing of the aqueous suspension of dispersed cells is discontinued cell aggregates are more likely than single cells to rapidly sink to the bottom (settle) of a culture vessel. The aggregate may consist of one cell type or may comprise different cell types. The constitution of the aggregate may be constant or may change. For example, initially the aggregate may predominantly consist of pluripotent stem cells whereas, during the culturing of the cells, (part) of the pluripotent stem cells may differentiate towards one or more pre-selected cell types, for example cardiomyocytes (e.g. atrial and/or ventricular). In some embodiments, the cells introduced in the culture system for in vitro manufacture of the one or more preselected cell types are introduced in the form of aggregates. In some embodiments, the cells introduced in the culture system for in vitro manufacture of the one or more preselected cell types are not in the form of aggregates and/or are, preferably, in the form of single cells. In this preferred embodiment of the method disclosed herein, the aggregates form during the mixing of the culture medium in the culture vessel. In a further preferred embodiment of the method, in the method, the pluripotent stem cells are introduced in the form of a single cell suspension, cultivated in culture medium for proliferation of the pluripotent stem cells (with no, one or more culture medium replacements according to the invention), thereby allowing the pluripotent stem cells to proliferate and to form aggregates in the culture vessel, and subsequently cultivated in culture medium for differentiation of the pluripotent stem cells towards the pre-selected cell type or cell-types (with no, one or more culture medium replacements according to the invention). In particular in such embodiments of the method of the invention desirable results can be obtained (e.g. with respect to the amount, relative amount, ratio of introduced cells versus obtained cells, and the like).

As used herein, “preselected cell type” refers to a cell of a certain type that was preselected as the cell type to be obtained with the method as disclosed herein. Such preselected cell type may, for example be a cardiovascular cell, a cardiomyocyte, an endothelial cell, a cell of the hematopoietic linage, a hematopoietic progenitor cell, a cell differentiated from a hematopoietic progenitor cell, a monocyte, a (common) myeloid progenitor, a (common) lymphoid progenitor, a macrophage, a T-cell, a B-cell, a NK-cell, a dendritic cell, a neuronal cell, a retinal cell, a lung cell, a liver cell, a pancreatic cell, or a cell belonging to the hemogenic endothelium. The term “preselected cell type” includes any “preselected cell type” lineage cells, and can be taken to apply to cells at any stage of the “preselected cell type” ontogeny, unless otherwise specified. For example, the “preselected cell type” may include both “preselected cell type” (lineage-restricted) precursor or progenitor cells (not being pluripotent stem cells) (i.e. cells that are capable, without dedifferentiation or reprogramming, of giving rise to progeny that include the “preselected cell type”, e.g. immature “preselected cell type” cells or fetal “preselected cell type” cells) and mature “preselected cell type” cells (adult-like “preselected cell type” cells). Preferably the “preselected cell type” cells are fetal, immature or mature (adult-like) “preselected cell type” cells. Such cells of the “preselected cell type” may express markers typical of the “preselected cell type” lineage and are well known in the art. The “preselected cell type” cells according to the invention are obtained in vitro from pluripotent stem cells by differentiation. The in vitro differentiation is done by means of a method as disclosed herein. The term “pre-selected cell type” may also refer to more than one preselected cell type obtained with the method as disclosed herein. For example, in certain embodiments the “pre-selected cell type” may refer to a T cell as well as to a lymphoid progenitor cell, or may refer to an atrial cardiomyocyte and a ventricular cardiomyocyte. Preferably the pre-selected cell type is a human pre-selected cell type.

For example, as used herein, “cardiomyocytes” or “cardiac myocytes” refer to any cardiomyocyte lineage cells, and can be taken to apply to cells at any stage of cardiomyocyte ontogeny, unless otherwise specified. For example, cardiomyocytes may include both cardiomyocyte precursor or progenitor cells (not being pluripotent stem cells) (i.e. cells that are capable, without dedifferentiation or reprogramming, of giving rise to progeny that include cardiomyocytes, e.g. immature cardiomyocytes or fetal cardiomyocytes) and mature cardiomyocytes (adult-like cardiomyocytes). Cardiomyocytes include atrial type cardiomyocytes, ventricular type cardiomyocytes, and nodal type cardiomyocytes. Preferably the cardiomyocytes are fetal, immature or mature (adult-like) cardiomyocytes. The cardiomyocyte progenitors, like the mature cardiomyocytes, may express markers typical of the cardiomyocyte lineage, including, without limitation, cardiac troponin I (cTnI), cardiac troponin T (cTnT), sarcomeric myosin heavy chain (MHC), GATA-4, Nkx2.5, N-cadherin, β1-adrenoceptor (β1-AR), ANF, the MEF-2 family of transcription factors, creatine kinase MB (CK-MB), myoglobin, or atrial natriuretic factor (ANF). The cardiomyocytes according to the invention are obtained in vitro by differentiation of pluripotent stem cells. The in vitro differentiation is done by means of a method as disclosed herein.

Likewise, “endothelial cells” refers to endothelial cells at any developmental stage, from progenitor to mature. The endothelial cells refer to a thin, flattened cell, of which a layer of the cells lines the inside surfaces of body cavities, blood vessels and lymph vessels, making up the endothelium. The endothelial progenitors, like the mature endothelial cells, may express markers typical of the endothelial lineage, including, without limitation, CD31, CD144 (VE-CADHERIN), CD54 (I-CAM1), vWF, VCAM, CD106 (V-CAM), VEGF-R2 (see e.g. Orlova et al. Arteriosclerosis, Thrombosis, and Vascular Biology. 2014; 34:177-186). The endothelial cells according to the invention are obtained from in vitro differentiated pluripotent stem cells. The in vitro differentiation is preferably done by means of a method as disclosed in the examples.

Likewise, hematopoietic lineage cells refer to any hematopoietic lineage cells, and can be taken to apply to cells at any stage of the hematopoietic ontogeny, including progenitors, unless otherwise specified. Hematopoietic progenitor cells (HPC) refers to a cell that remains mitotic and can produce more progenitor cells or precursor cells or can differentiate to an end fate hematopoietic cell lineage. Human markers for HPCs include: CD31, CD34, CD43, CD133, CD235a, CD41 and CD45, wherein CD41+ indicates megakaryocyte progenitors, CD235a+ erythrocyte progenitors, CD34+CD45+ early lymphoid/myeloid lineage progenitors, CD56+NK lineage progenitors, CD3+ T-cells, and CD19+CD20+ B-cells.

Likewise, neuronal lineage cells refer to any neuronal lineage cells, and can be taken to apply to cells at any stage of the neuronal ontogeny, including progenitors, unless otherwise specified. Neural progenitor cells (NPC) refers to a cell that remains mitotic and can produce more progenitor cells or precursor cells or can differentiate to an end fate neuronal cell lineage. Human markers for NPCs include: Sox2, Pax6 and Nestin. Mature neurons are positive for neuronal nuclei (NeuN), tubulin beta 3 class III (TUBB3) and microtubule-associated protein 2 (MAP2).

As used herein, “closed culture system” refers to a culturing system comprising a culture vessel and added components that is closed/sealed. Said closed- and/or sealed system typically undergoes sterilization prior to use and after being sealed, thus retaining its sterility. During the use of the culture vessel the integrity of the system is only minimally, preferably not, breached, thus maintaining the sterility of the system. The integrity of the system can for example be breached by lifting a cap or a lid, opening a valve or a tube, and the like). As used herein the term “closed system” preferably refers to a closed culturing system comprising a culture bioreactor or culture vessel and its components, including, for example, means for mixing culture medium comprised in the culture vessel and means for collecting and replacing (part of the) medium without breaching sterility. Said bioreactor is used to manufacture, maintain, culture, grow, differentiate, and manipulate a cell culture without a breach of the integrity of the sterility of the closed system. The closed system used in the method as disclosed herein allows for the collection and replacement of culture medium and/or (single cells) in the medium. Samples of culture medium may also be collected during cultivation/manufacturing of the cells in the closed culture system for in-process collection and analysis. Bags with media can be attached to the system using sterile connectors or using sterile tube welding (e.g. welders such as SOD® IIB terumo, biowelder Satorius and/or connectors such as kleenpak presto sterile connector (pall), Lynx S2S by Millipore, Opta SFT-1 by Sartorius Stedim Biotech, ReadyMate DAC by GE, or Pure-Fit SC by Saint-Gobain)

As used herein, “culturing”, “cultivating”, “growing” or variations thereof refer, when directed to a cell or cells, to a method step to propagate, expand or maintain a population of cells in culture media of various kind. Conventional methods and techniques are well-known to the skilled person in the field of molecular biology, biology, biochemistry, genomics, cell culturing and the like. Although the term “culturing” is generally understood to include the proliferation or division of cells, it also includes methods of differentiating cells in culture medium. As used herein, the term also included the purpose of the in vitro manufacture of the pre-selected cell type differentiated from pluripotent stem cell with the method as disclosed herein.

The term “culture media” also, and preferably, includes media that are suitable for the in vitro cell culture of human or animal cells for a prolonged period of time. Such culture media comprises sufficient components to allow the cells to grow, proliferate and/or differentiate over a longer period of, for example at least a day, preferably at least two, three, four, five, six, or more days. A “defined culture medium” refers to a (growth) medium suitable for the in vitro cell culture of human or animal cells and in which all of the chemical components are known. Such defined media does not or essentially not comprise any ill-defined source of nutrients and/or other ill-defined factors. A culture medium may, preferably, be serum-free. The culture media as described herein may comprise one or more compounds that are purposely included to steer proliferating and/or differentiation, i.e. compounds that are included in the culture medium and that, by contacting the cell in the culture vessel with the culture medium, e.g. for the duration of the contacting, steer e.g. differentiation during a particular stage of the differentiation of the cells towards the preselected cell type or cell types, for example by agonizing or antagonising particular (metabolic) pathways in these cells.

As used herein, “culture vessel” refers to a bioreactor, a tank, a flask or any other device suitable for the culturing of cells. The volume of the culture vessel as used herein can be any volume ranging from a few mL to hundreds of liters, preferably the culture vessel is between 2-150 liters, or between 2-100 liters, or between 2-50 liters in volume and/or allows for cultivation in such volumes of culture medium. Preferably the volume of the culture vessel is at least 2, 3, 5, 8, 10, 20, 50 liters in volume and/or has a volume that allows for cultivation in at least 2, 3, 5, 8, 10, 20, 50 liters of culture medium. As provided herein the culture vessel can have different configurations. In other words, the vessel can either be a vertical vessel, a vertical wheel reactor or a bag reactor or any other bioreactor known to the skilled person.

As used herein, “differentiating” and “differentiation” relate to the progression of a cell further down a developmental pathway within a lineage. Differentiation of pluripotent stem cells can be induced by means of compounds present in the culture medium and that direct differentiation of such stem cells within a lineage. As explained above, different (combination) of compounds, herein also referred to as steering compounds, are implied at different stages of differentiation of cells. Differentiation typically is controlled by the interaction of cellular genes and the chemical and physical surroundings of the cell, usually by means of signaling pathways involving proteins embedded in the cell surface. Alternatively, differentiation might be further steered by ectopic expression of genes that induce differentiation.

In the present invention differentiation is the biological process that pluripotent stem cells undergo in progressing towards a terminally differentiated cells within a cell-lineage. Effective differentiation processes are characterized by a high differentiation efficiency (number of cells that express the markers of the cells of interest; i.e. of the preselected cell type) and a high yield (number of cells obtained in the process). In order to obtain such high yield of the preselected cell type, it was surprisingly found that it is beneficial to have concomitantly differentiation and proliferation in the same process. It was also surprisingly found that, in some embodiments, it is beneficial in the method of the invention, that the pluripotent stem cells are introduced in the form of a single cell suspension, cultivated in culture medium for proliferation of the pluripotent stem cells (with no, one or more culture medium replacements according to the invention), thereby allowing the pluripotent stem cells to proliferate and to form aggregates in the culture vessel, and subsequently cultivated in culture medium for differentiation of the pluripotent stem cells towards the pre-selected cell type or cell-types (with no, one or more culture medium replacements according to the invention). In particular in such embodiments of the method of the invention desirable results can be obtained (e.g. with respect to the amount, relative amount, ratio of introduced cells versus obtained cells, and the like).

The process of differentiation towards a preselected cell type with the method according to the invention is induced in pluripotent stem cells, preferably of a human origin, by means of exposure to differentiation-inducing culture media compositions and using the method as disclosed herein. (Pluripotent) stem cells, can differentiate into any of the three germ layers, ectoderm, endoderm and mesoderm and can be further differentiated into cell types that are lineage-restricted progenitor cells, which in turn can differentiate into a more specific type of cell. Such lineage restricted-progenitor cells in turn can differentiate to further restricted cells (e.g., cardiac progenitors, endothelial progenitors, neural progenitors, lung progenitors, pancreatic progenitors, hematopoietic progenitors and the like), which in turn can differentiate into terminally differentiated cells (e.g. cardiomyocytes, endothelial cells, neurons, astrocytes, hepatocytes, alveolar cells, T-cells, B-cells, NK-cells, macrophages, erythropoietic cells and the like). Differentiation in general can be detected by the use of specific differentiation markers. Within context of the invention the (human) pluripotent stem cells are preferably differentiated into the preselected differentiated cell types and that display a fetal, but preferably mature or adult-like phenotype. The pluripotent stem cell is preferably an induced (human) pluripotent stem cell or an embryonic stem cells, preferably a human pluripotent stem cell. Preferably the pluripotent stem cell is a human pluripotent stem cell.

In addition to the afore mentioned pluripotent stem cells, also adult stem cells may be used in the method as disclosed herein. Adult stem cells include, for example, hematopoietic stem cells (HSCs), mammary stem cells, intestinal stem cells, mesenchymal stem cells, endothelial progenitor cells, endothelial progenitor cells, neural stem cells, olfactory adult stem cells, neural crest stem cells, and testicular stem cells (germ cells, spermatogonial stem cells). Therefore, according to another aspect, the invention disclosed herein with respect to pluripotent stem cells, also applies to the use of adult stem cells.

As used herein, “embryonic stem cells”, abbreviated as ‘ES cells’ or ESC (or if of human origin ‘hES cells’ or ‘hESCs’) refers to stem cells that are derived from the inner cell mass of a blastocyst. The skilled person understands how to obtain such embryonic stem cells, for example as described by Chung (Chung et al (2008) Stem Cell Lines, Vol 2(2):113-117), which employs a technique that does not cause the destruction of the donor embryo(s). Various ESC lines are listed in the NIH Human Embryonic Stem Cell Registry.

As used herein, “induced pluripotent stem cell” or “iPSC” refers to pluripotent stem cells that are derived from a cell that is not a pluripotent stem cell (i.e., from a cell this is differentiated relative to a pluripotent stem cell). Induced pluripotent stem cells can be derived from multiple different cell types, including terminally differentiated cells. Induced pluripotent stem cells generally have an embryonic stem cell-like morphology, growing as flat colonies with large nucleo-cytoplasmic ratios, defined borders and prominent nuclei. In addition, induced pluripotent stem cell may express one or more key pluripotency markers known by one of ordinary skill in the art. To generate induced pluripotent stem cells, somatic cells may, for example, be provided with reprogramming factors (e.g. Oct4, SOX2. KLF4, MYC, Nanog, Lin28, etc.) known in the art to reprogram the somatic cells to become pluripotent stem cells.

As used herein, “pluripotency” refers to an attribute of a (stem) cell that has the potential to differentiate into all cells constituting one or more tissues or organs, for example, any of the three germ layers: endoderm (e.g. interior stomach lining, gastrointestinal tract, the lungs), mesoderm (e.g. heart, muscle, bone, blood, urogenital tract), or ectoderm (e.g. epidermal tissues and nervous system).

As used herein, “pluripotent stem cell” or “PSC” refers to a stem cell capable of producing all cell types of the organism and can produce cells of the germ layers, e.g. endoderm, mesoderm, and ectoderm, of a mammal and encompasses at least pluripotent embryonic stem cells and induced pluripotent stem cells. Pluripotent stem cells can be obtained in different ways. Pluripotent embryonic stem cells may, for example, be obtained from the inner cell mass of an embryo. Induced pluripotent stem cells (iPSCs) may be derived from somatic cells. Pluripotent stem cells may also be in the form of an established cell line. Pluripotent stem cells might carry genetic manipulations to make the cells more suitable for cell therapy. For example, the cells might be edited in the HLA class I and II loci to become immune privileged. The cells might carry antigen receptors for targeting to a certain cell types. The cells might carry (inducible) constructs to promote differentiation to the desired cells cell types or carry inducible construct to kill the cells as a post-transplant safety measure.

As used herein, “proliferating” and “proliferation” relate to an increase (growth) in the number of cells in a population by cell division, i.e. cells undergoing mitosis. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation./pct

As used herein, “stem cells” refer to a population of undifferentiated cells defined by their ability at the single cell level to both self-renew and differentiate to produce progeny cells, including self-renewing progenitors, non-renewing progenitors, and terminally differentiated cells (Morrison et al. (1997) Cell 88:287-298). Stem cells have the ability to divide for indefinite periods in culture. Stem cells are cells that may be stably multiplied and cultured in vitro and are totipotent, pluripotent, induced pluripotent, multipotent, oligopotent, or unipotent cells, in the method disclosed herein the stem cells are preferably (at least) pluripotent, however, according to other aspects of the invention, it is also contemplated the stem cells for use in the methods as disclosed herein is a multipotent, oligopotent or unipotent stem cell, preferably a multipotent stem cell. Stem cells are categorized as somatic (adult) stem cells or embryonic stem cells. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

As used herein “undifferentiated” refers to a stem cell, that not yet has developed the characteristics of a further differentiated lineage-restricted progenitor. The terms undifferentiated and differentiated are, as will be appreciated by the person skilled in the art, relatively opposed to one another. Differentiated and undifferentiated cells are distinguished from each other by criteria that are well-established in the field, such as but not limited to morphological characteristics (e.g. size, shape, volume, ratio of nuclear volume to cytoplasmic volume), expression characteristics (e.g. presence of (genetic) markers) and the like.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to a new and surprising in vitro method for the manufacturing of a preselected cell type. The method is for inducing differentiation of pluripotent stem cells towards such preselected cell type and/or for manufacturing of such pre-selected differentiated pluripotent stem cell derived cell (or different types of cells), preferably in a closed culture system. The method allows vast amounts of such differentiated cells to be manufactured. The method allows for high output of cells over input of cells ratio's (e.g. expressed by number of cells).

The invention takes advantage of the fact that unlike most cells grown in bioreactors, pluripotent stem cells (as well as most adult stem cells) may form aggregates during suspension culture and do not require support like micro carriers. The invention can provide for a semi-automatic culturing method to obtain large amounts of differentiated cells obtained from pluripotent stem cells (by inducing differentiation). In particular it was found that a method can be provided that allows for the large scale production of a wide variety of pluripotent stem cell derived differentiated cells and which method is reliable, reproducible, and that does not rely on complex culturing steps and/or culturing devices. With the method as disclosed herein it is possible to produce vast amounts of differentiated cells and this manufacture of the preselected cell types from stem cells may be done in a relative short period of time and using a relatively simple and straight-forward methodology, therewith answering a real need in the field directed to the in vitro production of (human) differentiated cells derived from pluripotent stem cells.

The present invention provides hereto a method for the in vitro manufacture of a preselected cell type differentiated from a pluripotent stem cell, preferably in a closed culture system, wherein the method comprises the steps of:
a) providing pluripotent stem cells and a culture medium;
b) introducing the pluripotent stem cells and the culture medium into a culture vessel, preferably wherein the culture vessel is part of a closed culture system, wherein the culture medium is
i) a culture medium for proliferation of the pluripotent stem cells; or
ii) a culture medium for inducing differentiation of the pluripotent stem cells towards the pre-selected cell type;
c) mixing the culture medium in the culture vessel thereby allowing the cells to grow in the form of cell aggregates and preventing settling of the cell aggregates;
d) discontinuing the mixing of the culture medium in the culture vessel thereby allowing the cell aggregates to settle;
e) collecting part of the culture medium in the culture vessel;
f) optionally, in case in step b) a culture medium for proliferation of the pluripotent stem cells was used, introducing a further culture medium for proliferation of the pluripotent stem cells in the culture vessel and repeating step c)-e);
g) introducing a subsequent culture medium into the culture vessel, wherein the culture medium is a culture medium for inducing differentiation of the cells towards the pre-selected cell type;
h) mixing the culture medium in the culture vessel thereby allowing the cells to grow in the form of cell aggregates and preventing settling of the cell aggregates;
i) discontinuing the mixing of the culture medium in the culture vessel thereby allowing the cell aggregates to settle;
j) collecting part of the culture medium in the culture vessel and repeating steps g)-i) for a subsequent culture medium, or collecting (part of) the culture medium in the culture vessel, collecting the cell aggregates in the culture vessel, or collecting both.

It was surprisingly found that in the steps of the method wherein culture medium is removed or collected, only a part of the culture medium in the culture vessel may be removed before introducing the subsequent culture medium into the culture vessel. In other words, it was surprisingly found there is no need for removal of substantially all medium before providing new medium to the cells. Until now it has been the general understanding of the person skilled in the art that, in order to let differentiation protocols work, substantially all culture medium needs to be replaced before introducing the subsequent culture medium. In particular, such complete replacement of culture medium is considered necessary in differentiation protocols that rely on the use of different media comprising compounds that switches differentiation pathways on or off, such as it is the case for canonical Wnt signaling during cardiac differentiation of pluripotent stem cells. Contrary to this general understanding, it has now been found that it is not required that substantially all culture medium is replaced before introducing a subsequent culture medium, which subsequent culture medium may have a different composition, for example by comprising more or less of different nutrients that, for example, direct the further differentiation of the cells towards the preselected cell type. In fact, it is the inventor's observation that by leaving at least (a substantial) part of the previous medium in the culture vessel this not only avoids additional steps that can be harmful to the health of the cells but also provides for improved survival, cell number and properties of the thus obtained differentiated stem cells.

Without being bound by theory, the inventors contemplate that the surprising effects obtained with the method of the invention are at least in part due to the fact that with the method as disclosed herein, proliferation and/or differentiation of the cells towards the desired pre-selected cell type(s) may continue undisturbed. It is believed by the inventors that prior art methods that require such handling of the cells like spinning down (pelleting) of cells to remove culture medium, filtering of the cells to remove culture medium, washing of cells to remove culture medium, or that include continuous removal and replacement of culture medium may lead to less optimal proliferation and differentiation of the cells.

In particular differentiation towards a particular differentiated cell consist of a delicate and balanced involvement of different (signaling) pathways that need to be switched-on or switched-off at different stages of differentiation (e.g. by using particular agonists or antagonists of the pathways) and the inventors contemplate that by removing a first medium from the cells (i.e. separating cells and medium) before providing a second medium to the cells, or by washing the cells, and so on, this may cause a (temporary) slowing-down, interruption, or even disturbance of the ongoing differentiation of the cells. The inventors believe that by not removing substantially all of the culture medium, the process of differentiation remains undisturbed, or is at least disturbed to a lesser extent. In addition, it is believed by the inventors that by allowing part of the culture medium to remain in the culture system, additional factors produced by the cells during in particular differentiation contribute to the continued differentiation of the cells. It is believed by the inventors that the above may explain, at least in part, the surprising high number of cells that are obtained with the method of the invention.

In addition, in a preferred embodiment, in the method of the invention, the pluripotent stem cells are introduced in the culture vessel in the form of a single cell suspension and allowed to form aggregates while culturing. In a preferred embodiment, the cells introduced in the form of a suspension of single cells are allowed to form aggregates while being cultivated in a culture medium for proliferation of the induced pluripotent stem cells. In another embodiment, the cells introduced in the form of a suspension of single cells are allowed to form aggregates while being cultivated in a culture medium for differentiation of the induced pluripotent stem cells. In one embodiment, the cells in the single cell suspension are introduced in the culture vessel in a culture medium for proliferation of the pluripotent stem cells. Without being bound by theory, it is believed that by introducing the pluripotent stem cells in the culture vessel in the form of a suspension of unaggregated cells, and allowing aggregate formation to take place during the subsequent cultivation in the culture vessel, an optimal and homogenous population of cell aggregates are obtained in the culture vessel and that provide for improved manufacturing of the pre-selected cell type.

It is thus found it is possible to manufacture differentiated cells (of the preselected cell type) from pluripotent stem cells, preferably using closed culture systems, without the need of complicated centrifugation steps, and wherein the pluripotent stem cells that are provided to the closed culture system are, optionally proliferated, differentiated towards the preselected cell type in one and the same culture system in a manner that allows for the production of vast amount of such differentiated cells.

In addition it was found that the method as disclosed herein is robust in that it can be applied to obtain a wide-variety of differentiated cells. Surprisingly it was found that the method is robust enough to allow optimization at small scale (e.g. 15 mL) and subsequent upscaling to cultivation using multiple liters of medium. It was surprisingly found that with the method as disclosed herein it becomes possible to upscale manufacturing of preselected cell types by differentiation of stem cells in culture vessels with increased volume. The method as disclosed herein allows for highly efficient manufacturing of the preselected cell type from stem cells and provides high output over input ratios with respect to the number of cells obtained by the method as disclosed herein versus the amount of initial stem cells provided and allows for high cell densities. For example in some embodiments the initial stem cell density is about 200000 cells per milliliter of culture medium, whereas the final density of a particular pre-selected cell type obtained by the method as disclosed herein can be 3 000 000 cells or higher per milliliter of culture medium (a factor or ratio of, in this example, 15 or higher). For example, in some embodiment the ratio between the number of cells introduced and the number of pre-selected cells obtained is at least 1:10, 1:12, 1:15, 1:20, or 1:25. Such numbers are very desirable but appear unprecedented in view of the prior art with respect to the preselected cell type. With the method as disclosed herein it has now become possible to manufacture higher number of the preselected cell types, at higher densities of cells per volume of medium, and in larger volumes of culture medium, using simple, reproducible method steps, which steps are robust enough to be applicable to the manufacture of a wide range of preselected cell type from pluripotent stem cells and therefore provide for a long felt need in the field.

In addition, with the method as disclosed herein it is possible to easily control culturing parameters like pH, CO2, biomass, dissolved oxygen, and lactate concentration. Such parameters can, in turn be used, in combination with the used differentiation protocols to obtain the preselected cell type, to increase or optimize cell density during the cultivation with the purpose of maximizing the yield of the preselected cell type. For example, based on biomass, cell density and/or lactate buildup in the culture medium during cultivation, the most optimal moment of medium collection and fresh medium addition can be determined, further optimizing the overall production of the preselected cell type.

By providing the method as disclosed herein, and wherein the population of cells, i.e. the cell aggregates, is allowed to settle between (each) culture medium collection steps, the culture medium present in the culture vessel can be easily partially collected, e.g. by using suction from the upper part of the medium downwards, while leaving the population of cell aggregates cells and part of the culture medium unaffected. After the addition of a subsequent (and preferably different in composition) culture medium, the method of inducing differentiation of cells is continued by mixing the population of settled cells.

One unique advantage of this approach is that the system also allows for harvesting of single cells, and proteins and/or exosomes secreted from aggregates in the culture system. To enable this, mixing of the medium in the culture vessel is discontinued for a period of time and settling of the cells is performed for sufficient time to keep most of the single cells or the proteins and/or exosomes in suspension in the medium but to allow for settling of the much larger and heavier aggregates. Next, medium including the single cells, secreted proteins and or exosomes is harvested by suction. This is particularly useful for cells of the hematopoietic lineage which are formed by the hemogenic endothelium and which are secreted as hematopoietic progenitor cells (HPC) from the cell aggregate as single cells in the culture medium. The hematopoietic progenitor cells (HPC) can produce more progenitor cells or precursor cells or can differentiate to an end fate hematopoietic cell lineage cell such as a macrophage, dendritic cell, T-cell or B-cell. This differentiation may be performed in the same culture vessel or may be performed, after harvesting of the hematopoietic progenitor cells, in a further culture system.

Although for proliferating stem cells settling of aggregates was found to be associated with a high risk of undesirable aggregate conglomeration/fusion because aggregates are concentrated together at the bottom surface, or the like, no such deficiencies were observed by the inventors in the method of the present invention, and wherein the stem cells are induced to differentiate towards a pre-selected cell type and wherein the culture medium is only partially replaced. In fact, it was found that by using the method of the present invention, the population of preselected cell type cells obtained have excellent cell characteristics and cell properties, such as (expression) markers or biological activity.

It is further noted that the method of the present invention facilitates the differentiation and, optionally as a first phase of the method, proliferation, of cell aggregates of pluripotent stem cells, preferably in a continuously closed culture system. Without the need of any (extensive)(manual) human intervention, the present invention provides a method for manufacturing a preselected cell type by inducing differentiation of pluripotent stem cells in a closed culture system with a reduced risk of cross contamination.

The closed culture system allows for a method as disclosed herein wherein cells can be cultivated, cultured, grown for a prolonged period of time. In said closed systems pluripotent stem cells are allowed to form cell aggregates and to differentiate towards the preselected differentiated cells. The system allows for a sterile system, reduces human intervention, thus reducing the risk of cross contamination.

The method as disclosed herein comprises the step of a) providing pluripotent stem cells and a (first) culture medium and b) introducing the pluripotent stem cells and the culture medium into a culture vessel, and that is preferably part of a closed culture system. Alternatively, the culture medium and/or the stem cells are already present in the culture vessel and either the pluripotent stem cells or the culture medium is added to the culture vessel.

The culture medium may be
i) a culture medium (suitable) for proliferation of the pluripotent stem cells; or
ii) a culture medium (suitable) for inducing differentiation of the pluripotent stem cells towards the pre-selected cell type. Preferably the culture medium provided in step a) and introduced in step b) is a culture medium for inducing differentiation of the pluripotent stem cells towards the pre-selected cell type(s).

For example, depending on the number of pluripotent stem cells introduced, it may be decided to first allow the pluripotent stem cells to proliferate for a certain period of time in order to obtain a desirable number of pluripotent stem cells (aggregates). In such case, the culture medium may be any suitably a culture medium for proliferation of the pluripotent stem cells, such as commercially available mTeSR1, StemMACS™ iPS-Brew XF, Essential 8, TeSR E8, mTeSR Plus and/or Nutristem media. In (preferred) embodiments wherein the pluripotent stem cells are introduced as a single cell suspension, the single cells will form aggregates during cultivation in the culture medium for proliferation of the stem cells (aggregation will typically start within a few hours, for example after 2-3 hours).

Alternatively, for example in case a sufficient number of pluripotent stem cells are introduced into the culture vessel (e.g. 0.5-1.5 million cells, or more, per ml), differentiation of the pluripotent stem cells towards the preselected cell type may immediately be induced by adding as a first culture medium a culture medium that is suitable for initiating or inducing differentiation of the pluripotent stem cells towards the preselected cell type. In (preferred) embodiments wherein the pluripotent stem cells are introduced as a single cell suspension, the single cells will form aggregates during cultivation in the culture medium for inducing differentiation.

Depending on the pre-selected cell type to which the pluripotent stem cells needs to be differentiated the skilled person knows what kind of culture medium can suitable be used. In that respect it is noted that it is not required that the culture medium provides for full differentiation of the pluripotent stem cell to the preselected cell type, but it may also be, as is explained herein, that culture media with different consecutive compositions are used to initiate, steer, promote and/or enhance differentiation of the cells to the pre-selected (differentiated) cell type.

With regard to the individual steps as defined in the method of the present invention, it is noted that in step a) any kind of pluripotent stem cells, including induced pluripotent stem cells, known to the skilled person may be provided to the culture system. For example, although the pluripotent stem cells may be provided as a population of pluripotent stem cell aggregates, it is also possible, as an alternative, to provide pluripotent stem cells as single cells (e.g. dispersed in a suitable medium), and allowing these cells to form aggregates once present in the culture vessel. The pluripotent stem cells provided may be of one type or may be a mixture of different types of pluripotent stem cells.

As already mentioned above, it is noted that the (further) proliferation of cell aggregates may be facilitated by a proliferation medium provided to the closed culture system before providing a medium facilitating the differentiation of the cell aggregates in the culture vessel.

The cells provided in step a) can be provided in an inoculum comprising single cells, aggregates or both, such that the initial cell density of the pluripotent stem cells in the culture vessel during before cultivation is initiated in step c) is preferably between 1×10⁴-1×10⁶ cells per milliliter of culture medium.

The pluripotent stem cells of step a) are preferably pre-cultured in, for example, cultivation flasks or bioreactors, prior to provision in step a) of the method. Prior to step a) the cells may be, for example, be cultured in a culture medium suitable for culturing (human) pluripotent stem cells, for example (human) induced pluripotent stem cells (iPSCs) or (human) embryonic stem cells (ESCs). Prior to provision in step a) cells may be washed and may be subsequently dissociated from each other and/or the culturing flask.

Furthermore, the culture medium provided in step a) may already contain the single cells and/or aggregates of pluripotent stem cells or the cells may be added to the culture medium once the culture medium is in the, preferably closed, culture system.

In step b) of the method as disclosed herein, the culture medium and the pluripotent stem cells are introduced in a culture vessel, preferably that is part of a closed culture system. The cells that are introduced in the culture vessel according to step b) as disclosed herein are the cells provided in step a). Similarly, the culture medium introduced in the culture vessel according to step b) is the culture medium as provided in step a).

There are many types of closed culture systems, culture vessels or bioreactors that are suitable for the method as disclosed herein. In particular the bioreactor as provided herein preferably contains a stirrer mechanism or other mean that allow for mixing of the culture medium comprising the cells during cultivation. Examples of suitable systems are custom stainless steel/glass reactors or single use systems such as Ambr and Biostat (Sartorius), PBS (PBS biotech), Dasbox and Bioflo (Eppendorf), Appiflex (Applikon), Wave and Xuri (GE/Cytiva) in a typical volume range of a few milliliters up to hundreds of liters.

Further, the bioreactor as disclosed herein may comprise controls for oxygen and CO2 and probes for measuring the biomass, cell density, pH-value of the culture medium, lactate concentration, and/or for measuring the amount of dissolved oxygen contained in the culture medium, as well as introduced in the culture vessel. Such probes and controls are known to the skilled person.

After the suitable culture medium and pluripotent stem cells are introduced into the culture vessel cultivation is started in step c) of the method as disclosed herein. As will be understood the cultivation of the cells is performed in a, preferably suitable buffered, culture medium, at a temperature that is suitable for cultivation of the cells and/or at a temperature that is suitable for proliferation or differentiation, depending on whether the culture medium is i) a culture medium for proliferation of the pluripotent stem cells, or ii) a culture medium for inducing differentiation of the pluripotent stem cells towards the preselected cell type.

Parameters such as pH, temperature, dissolved oxygen concentration and osmolarity of the cell culture medium will depend on the type of cell as a skilled person understands. The person skilled in the art knows how to provide for optimal pH, temperature, dissolved oxygen concentration and osmolarity.

Preferably, for the method as disclosed herein, during cultivation, the pH is chosen between 6.9 and 7.5 and/or the temperature is chosen between 29 and 39° C. and/or the osmolarity is chosen between 260 and 400 mOsm/kg.

In another embodiment, a bicarbonate buffered basal medium, using online pH measurements is used. PH adjustments can be made using CO2 for lowering the pH and base (bicarbonate or NaOH) to increase pH. Likewise, oxygen is preferably controlled to stay within physiologically relevant ranges (3-21%).

Step c) of the method provided herein comprises the step of mixing the culture medium containing the cells as introduced in the culture vessel in step b). The mixing is typically induced by the mixing means of the culture vessel/culture system. Any kind of mixing means may be used, e.g. mixing by stirring the culture medium or mixing by (gently) shaking the culture vessel.

Preferably the mixing is continuously although it is also contemplated that mixing of the culture medium comprising the cells may be discontinued for a short period of time during step c) (and/or step h), for example for 1 to 5 minutes, before mixing in said step is continued.

The mixing of the culture medium allows the cells in the culture vessel to (continue to) grow in the form of cell aggregates (also in case pluripotent stem cells are initially introduced as single cells or as a cell suspension, in which case aggregates are formed during the cultivation). The mixing of the culture medium was also found to allow the (pluripotent stem) cells to differentiate towards the preselected cell type in the presence of a suitable culture medium for differentiation.

Further, the mixing of the culture medium prevents settling of the cell aggregates and/or adhering of the cell aggregates to each other. Depending on, for example, the number of cells in the culture medium, the culture medium used and so on, the skilled person understands how to mix the culture medium such that the cells are allowed to form or remain in the form of cell aggregates while at the same time preventing settling of said aggregates during the cultivation/growth.

The mixing of the culture medium, and consequently the cultivation of the cells in step c) of the method as disclosed herein may be as long as desired within the context of the current invention, and/or as long as the culture medium provided to the culture vessel support cultivation of the cells.

In one embodiment the mixing is continued until the culture medium in the culture vessel does no longer support growth of the cells in the culture vessel. In another embodiment the mixing is continued for a certain amount of time before the mixing is discontinued and the cells aggregates are allowed to settle (e.g. as provided in step d) of the method according to the invention). For example, the mixing in step c) may be continued for at least 1, 2, 4, 6, 8, 12, 24, 36, 48, 72, 96 hours, for example between 1-72 hours, 2-60 hours or 2-48 hours.

After the culture medium has been mixed for the desired amount of time to allow growth of the cells in the culture vessel in the form of cell aggregates, in step d) the mixing of the culture medium in the culture vessel is discontinued. By discontinuing the mixing of the culture medium, the cell aggregates in the culture medium are allowed to settle, preferably by gravity.

Although it is contemplated that any time period for discontinuation of the mixing of the culture medium in step d) may be selected, it was found that, depending on the configuration of the reactor one can easily determine the time needed to let the aggregates settle and/or to let the single cells, secreted proteins and exosomes float in the medium. Typically the time required to allow the cell aggregates to settle, i.e. to sink towards the bottom of the culture vessel is between 5 minutes and 240 minutes, preferably between 20 minutes and 60 minutes in a 3 liter bioreactor (vessel).

In step e) part of the culture medium is collected from the culture vessel, for example by (careful) suction of the culture medium. Although other culture medium discharging or collecting means may be used to collect part of the culture medium from the culture vessel, suction is the most preferred option. Collection of the culture medium from the culture vessel wherein the cell aggregates have settled is such that the culture medium is collected in a manner that the cell aggregates are the least disturbed and remain settled, for example by suction from the upper layers of the culture medium. Removal of part of the culture medium preferably is performed such that no or only limited aggregates are removed from the culture vessel.

Alternatively, instead of removing the culture medium by means of suction, the culture medium may also be drained from the culture vessel, using a drainage system, for example located at a predefined height away from the bottom of the culture vessel. Such height may, for example, depend on the thickness of the layer of the cell aggregates settled in the culture vessel and/or of the amount of culture medium that is to be removed.

Irrespective the way by which the culture medium is collected from the culture vessel, it is noted that it was surprisingly found that it is possible to design robust and reproducible manufacturing processes yielding well-differentiated cells in high quantities and using a preferably closed system by not collecting all of the culture medium from the culture vessel and replacing it with fresh culture medium, but by collecting only part of the culture medium from the culture vessel before a subsequent, fresh, culture medium is added to the remaining culture medium in the culture vessel (and thus wherein part of the previous medium is mixed with the subsequent medium).

For example, in one embodiment at most 95 vol. %, 90 vol. %, 85 vol. %, or 80 vol. % of the culture medium in the culture vessel is collected to enable efficient switching of media composition and/or harvesting of (single) cells or material secreted by the cells into the culture medium. For example, in this embodiment, in case the culture vessel comprises 10 liter of culture medium, at most 9500, 9000, 8500 or 8000 milliliter of the culture medium is collected from the culture vessel.

At the same time it was found that, preferably, at least 30 vol. %, 40 vol. % 50 vol. %, 60 vol. % or 70 vol. % of the culture medium is collected from the culture vessel. Therefore, in some embodiments between, for example between 30 vol. %-95 vol. %, or between 40 vol. % and 95 vol. % or between 50-95 vol. %, or between 60-90 vol. % or between 60-80 vol. %, or between 70-90 vol. % or between 70-80 vol. % of the culture medium is collected from the culture vessel in step e) (and/or in step j)). As will be understood by the skilled person, the amount or percentage of medium collected may vary between different moments of collection medium according to the method of the invention. For example, during a first medium collection 70 vol %. % of the culture medium in the culture vessel may be collected and replaced, whereas during a subsequent medium collection, the same, more or less (e.g. 50 vol. % or 75 vol. %) may be collected and replaced.

At the same time, it will be understood by the skilled person that the method of the invention may also include, for example after step h) or i), and preferably before step j) additional cultivation steps (one or more) and wherein, after step i) substantially all culture medium in the culture vessel is collected (e.g. more than 95 vol. %, preferably more than 98 vol. %) and replaced by a subsequent culture medium (same of different volume), followed by cultivating the cells in the subsequent culture medium (or media in case this step is repeated more than once), and, preferably, subsequently collecting the culture medium in the culture vessel, collecting the cell aggregates in the culture vessel, or collecting both.

It will also be understood by the skilled person that after, for example, having performed the method of the invention at least once until step h) or i), preferably at least two, three of more times, cultivation of the cell may be continued using other techniques of medium replacement/refreshing or culturing, such as, for example by perfusion of culture medium and wherein a certain (small) volume of culture medium is refreshed continuously or every hour of few (e.g. 2-5) hours. In other words, in such embodiments, cells are first obtained with the method according to the invention, and as described herein, and whereafter the cells are continued to be grown, for example, further differentiated, using common or other culture media replacement techniques or culture techniques such as perfusion (e.g. as is known in the art). The continued cultivation of the cells may occur in the same culture vessel or the cells or cell aggregates may first be collected and be introduced into a new culture device, system or vessel, and further cultivated therein. For example, in the case of macrophages, the method of the current invention can be used to obtain high number and quality of hemogenic endothelium cells and/or monocytes. Such cells may than be further differentiated towards macrophages either by continuing the use of the method as disclosed herein, for example in the same culture system/culture vessel, or by using common or other culture media replacement techniques or culturing techniques and methods, such as perfusion. It may also be decided to collect the cells (or aggregates) and transfer these to a new culture system and allow the transferred cells to continue to grow or differentiate towards, in this example, macrophages.

The collected culture medium may be discharged or may be used to, for example, isolate single cells present in said medium or to collect other materials that may be present is such medium, for example secreted proteins, hormones or, in preferred embodiments, exosomes or other types of extracellular vesicles.

Preferably the amount of culture medium that is removed from the culture vessel in step e) and/or step j) is as such that the ratio of culture medium that remains behind in the culture vessel and the fresh culture medium that is added to the culture vessel in step f) or step g) (as discussed below) is from 1:1 to 1:15, for example 1:3-1:10, for example 1:4-1:8, for example 1:1.5 or 1:2.5, or 1:5 With respect to the freshly added culture medium added after collecting part of the culture medium in the culture vessel, it is noted that the volume thereof may be different from the volume of culture medium that was collected. For example, in case 4 liter of culture medium is removed, the volume of fresh medium introduced into the culture vessel may be 4 liter, but also more than 4 liter or less than 4 liter. The volume of the fresh medium added to the culture vessel may, for example, be between 10-200%, for example 50-150% of the volume of the culture medium that was collected from the culture vessel. Consequently, the volume of medium after fresh medium is added may be the same, of may be less or more than the volume of the previous medium (for example in case the previous medium was 10 liter, and 7 liter was removed before 6 fresh medium was added, the volume of medium after fresh medium is added in 9 liter, which is 1 liter less than the volume of the previous medium).

In some preferred embodiment the process described in the application can be performed in combination with feed strategies to delay medium refreshment in the process. Feed strategies rely on adding highly concentrated nutrient mixes in a small volume to the bioreactor to compensate for nutrients being fully metabolized by the cells. These feed strategies allow to keep critical nutrients at physiologically relevant levels and can help to avoid toxicity caused by too high or too low concentrations of critical nutrients. Therefore, in some embodiments, the method includes the adding to the culture medium, e.g during step c) and/or step h) of additional components or nutrients. Preferably, in such embodiments the adding is performed without discontinuing mixing and/or without (partial) replacing of the medium present in the culture vessel.

After part of the culture medium is collected from the culture vessel, new fresh medium is introduced into the culture vessel to allow for another cycle of mixing and growth (proliferation and/or differentiation) of the cell aggregates that are present in the culture vessel.

As discussed above, in certain embodiments, and before differentiation is induced, it may be desirable to first allow the pluripotent stem cells introduced in step b) of the method as disclosed herein to proliferate, for example to increase the total number of cells in the culture vessel.

In such case, and as indicated above, a culture medium suitable for proliferation of the pluripotent stem cells is used in steps a)-e) discussed above. If so desired, for example to even further increase the total number of pluripotent stem cells in the culture vessel, steps c)-e) may be repeated by providing the cell aggregates in the culture vessel with a further culture medium for proliferation of the pluripotent stem cells (pluripotent stem cell aggregates) in the culture vessel. Such further proliferation medium may or may not be the same as the first proliferation medium.

However, if a further cycle of proliferation of the pluripotent stem cells is not required or desired this additional cycle of proliferation of the cells may be skipped. Accordingly there is provided for the optional step f) wherein in case in step b) a culture medium for proliferation of the pluripotent stem cells was used, a further culture medium for proliferation of the pluripotent stem cells is introduced in the culture vessel and after which steps c)-e) are repeated. A skilled person understands that in principle these optional proliferations steps may be repeated as often as is required and before the pluripotent stem cells are cultivated in a culture medium that induces differentiation of the cells towards the preselected (differentiated) cell-type. However, in practice it was found that repeating the steps c)-e) for further proliferation the pluripotent stem cells should be limited to no more than 3, preferably 2 of the cycles (steps c)-e)).

However, according to another embodiment, for example, and in a preferred embodiment, in case in step b) a culture medium for inducing differentiation of the pluripotent stem cell towards to preselected cell type is used, or in case in step b) a culture medium for proliferation of the pluripotent stem cells is used and no further proliferation using a culture medium for proliferation of the pluripotent stem cell is required or desired, in step g) fresh culture medium is added to the cell aggregates and the remaining previous culture medium in the culture vessel.

The subsequent culture medium that is added in step g) to the cells (and medium that remained after step e)) is a culture medium for inducing differentiation of the pluripotent stem cells towards the preselected cell type. Therefore, depending on the culture medium used in step b) of the method as disclosed herein, the cell aggregates are either now induced to differentiate towards the preselected cell type or are further steered towards the preselected (differentiated) cell type.

In step h) the cells are allowed to differentiate towards the preselected cell type under mixing of the culture medium for differentiation of the cells.

As already explained for step c), preferably the mixing is continuously although it is also contemplated that mixing of the culture medium comprising the cells may be discontinued for a short period of time during, for example for 1 to 5 minutes, before mixing in said step is continued.

The mixing of the culture medium allows the cells in the culture vessel to continue to grow, as well as differentiate, in the form of cell aggregates. The mixing of the culture medium was indeed found to allow the cells to differentiate towards the preselected cell type in the presence of a suitable culture medium for differentiation.

To the surprise of the inventors it was found that during differentiation of the cells towards the preselected (differentiated) cell type, the cell aggregates showed good and improved cell total cell counts at the end of the process (number of cells), which is indicative of continued growth in the number of cells during the cultivation under conditions that induce/steer differentiation towards the preselected cell type (yield). And the same time the method showed homogenous differentiation of the cells in the aggregates (thus providing a homogenous cell population of the preselected cell type).

In other words, with the method as disclosed herein it is possible to both improve yield of differentiated cells (preselected cell type) as well as quality of the obtained differentiated cells in that a large percentage of the cells differentiated towards to preselected cell type in a comparable matter, providing a relative homogenous population of differentiated cells, displaying comparable characteristics throughout the obtained cell population (preselected cell type).

In some embodiments, the mixing in step h) may be continued for at least 1, 2, 4, 6, 8, 12, 24, 36 or 48 hours, and for example up to 1 week or 2 weeks, for example between 12-48 hours, 2-60 hours or 2-96 hours.

After the culture medium has been mixed for the desired amount of time to allow growth and differentiation of the cells in the culture vessel, in step i) the mixing of the culture medium in the culture vessel is discontinued. By discontinuing the mixing of the culture medium, the cell aggregates in the culture medium are again allowed to settle, preferably by gravity.

As already explained above for step d), likewise for step i) it is contemplated that any time period for discontinuation of the mixing of the culture medium may be selected. It was found that, depending on the configuration of the reactor one can easily determine the time needed to let the aggregates settle and/or to let most of the single cells, secreted proteins and exosomes float in the medium. Typically the time required to allow the cell aggregates to settle, i.e. to sink towards the bottom of the culture vessel is between 5 minutes and 240 minutes, preferably between 20 minutes and 60 minutes in a 3 liter bioreactor (vessel). It was found that if settling was allowed to proceed too long (e.g. for more than 480 minutes) this may cause damage to the cells or cause increased cell death. Such long settling times are normally also not required, as will be understood by the skilled person.

In step j) part of the culture medium may collected from the culture vessel, in a manner as is already explained above for step e). Again, and irrespective the way by which the culture medium is removed from the culture vessel, it is noted that it was surprisingly found that it is possible to obtain well-differentiated cells in desirable amounts when not all of the culture medium is collected and replaced but when only part of the culture medium is collected from the culture vessel before a subsequent, fresh, culture medium is added to the culture medium (and thus wherein part of the previous medium is mixed with the subsequent medium).

In a preferred embodiment of step j) at most 95 vol. %, 90 vol. %, 85 vol. %, or 80 vol. % of the culture medium in the culture vessel is collected to enable efficient switching of media composition and/or harvesting of (single) cells or material secreted by the cells into the culture medium. For example, in case the culture vessel comprises 10 liter of culture medium, at most 9500, 9000, 8500 or 8000 milliliter of the culture medium is collected from the culture vessel. At the same time it was found that, preferably, at least 30 vol. %, 40 vol. %, 50 vol. %, 60 vol. % or 70 vol. % of the culture medium is collected from the culture vessel. Therefore, in some embodiments between, for example between 30 vol. %-95 vol. %, or between 40 vol. % and 95 vol. % or between 50-95 vol. %, or between 60-90 vol. % or between 60-80 vol. %, or between 70-90 vol. % or between 70-80 vol. % of the culture medium is collected from the culture vessel.

The collected culture medium of step j) may be discharged or may be used to, for example, isolate single cells present in said medium or to collect other materials that may be present is such medium, for example secreted protein, hormones of, in preferred embodiments, exosomes or other types of extracellular vesicles.

Preferably the amount of culture medium that is removed from the culture vessel in step j) is as such that the ratio of culture medium that remains behind in the culture vessel and the fresh culture medium that is added to the culture vessel in case steps g)-i) above are repeated is from 1:1 to 1:15, for example 1:3-1:10, for example 1:4-1:8 for example 1:1.5 or 1:2.5, or 1:5 With respect to the freshly added culture medium added, in case step g)-i) above are repeated, after collecting part of the culture medium in the culture vessel, it is noted that the volume thereof may be different from the volume of culture medium that was collected. For example, in case 4 liter of culture medium is removed, the volume of fresh medium introduced into the culture vessel may be 4 liter, but also more than 4 liter or less than 4 liter. The volume of the fresh medium added to the culture vessel may, for example, be between 10-200%, for example 50-150% of the volume of the culture medium that was collected from the culture vessel. Consequently, the volume of medium after fresh medium is added may be the same, or may be less or more than the volume of the previous medium (for example in case the previous medium was 10 liter, and 7 liter was removed before 6 fresh medium was added, the volume of medium after fresh medium is added in 9 liter, which is 1 liter less than the volume of the previous medium).

Steps g)-i) may be repeated as often as needed or desired, for example in case differentiation of the cells towards the preselected cell type requires the use of several different or the same culture media for differentiation, or for example, in case the preselected cells or other materials are obtained from the collected culture media (e.g. not from the aggregates as such), and multiple rounds of steps g)-i) and collecting of the culture medium allows to obtain increased number of such preselected cells that are present in the culture medium (for example secreted from the aggregates into the culture medium as single cells).

Once after step i) the obtained cells have differentiated sufficiently towards or into the preselected cell types the cell aggregates in the culture vessel and/or the culture medium in the culture vessel may be collected for further use, as already discussed above. Collecting and further handling of the cell aggregates of the culture medium may be performed by any method or procedure known to the skilled person and suitable for the purpose of the cell aggregates and/or culture medium.

In a preferred embodiment of the invention the method does not comprise a centrifugation step nor a filtration step during any of steps b)-j), except for possibly the collecting of the differentiated cells from the culture medium/cell aggregates in step j). A centrifugation step and/or a filtration step require(s) manual or complex intervention in the method for culturing (not collecting) of the cells during steps b)-j) and as such, preferably, excluded from the method as disclosed herein. Rather, the method as disclosed herein relies on gravitational force for settling of the cell aggregates (e.g. in combination with collecting only part of the medium during steps e) and/or j)).

In a preferred embodiment, steps g)-i) is repeated once or more than once, using one or more subsequent culture media. For example, steps g)-i) may be repeated one, two, three, four, five or more times. It was surprisingly found that the method according to the invention is both robust enough and gentle enough to allow several cycles of growing the cells, collecting culture medium as disclosed and adding new culture medium to allow for further growing and differentiation of the cells.

It was found that the cell aggregates remain largely intact, that differentiating cells in the cell aggregates remain highly viable and that differentiation of the cells commences in a relatively homogenous manner. In other words, the method as disclosed herein allows, by providing a method wherein steps g)-i) are repeated one or more times the further and better differentiation of the cells.

In some embodiments, by repeating step g)-i) using a subsequent culture medium for differentiation of the cells (which culture medium may or may not be the same as the prior culture medium for differentiation of the cells used), the further differentiation of the cells derived from the pluripotent stem cells is further facilitated.

Such repetition of differentiation cycles increased the quality of the differentiated cells to be obtained as well as the scalability of the present method.

In some embodiments, there is provided for a method as disclosed herein, wherein in step b) the pluripotent stem cells are introduced in the form of a single cell suspension or wherein in step b) the pluripotent stem cells are introduced in the form of cell aggregates. Indeed although preferably the pluripotent stem cells may be provided as a population of pluripotent stem cell aggregates, it is also possible, as an alternative, to provide pluripotent stem cells as single cells (e.g. dispersed in a suitable medium), and allowing these cells to form aggregates once present in the culture vessel. The pluripotent stem cells provided may be of one type or may be a mixture of different types of pluripotent stem cells.

In some embodiments, there is provided for a method as disclosed herein, wherein in step b) the amount of pluripotent stem cells (as single cells and/or as aggregates) in the culture medium is between 1×10⁴-1×10⁶ pluripotent stem cells per ml culture medium. In other words, in such embodiments, the initial cell density of the pluripotent stem cells in the culture vessel before cultivation is initiated in step c) is between 1×10⁴-1×10⁶ cells per milliliter of culture medium, preferably the cell density is between 5×10⁴-5×10⁵ cells per milliliter of culture medium.

It was found that in order to obtain high yield of the preselected cell type it is desirable that the cell density of the pluripotent stem cells should not be too high as this was found to impact yield and quality of the obtained cells. For example, in case the pluripotent stem cells are introduced in the form of single cells, the cell density in the culture medium must be such that the aggregates that initially form preferably have sizes as disclosed herein below, which may not be achievable when cell density is too high (or too low).

In another preferred embodiment of the method as disclosed herein the cell density of pluripotent stem cells before differentiation of the cells is induced in step c) or in step g) is between 1×10⁴-1×10⁶ cells per milliliter of culture medium, preferably the initial cell density is between 5×10⁴-5×10⁵ cells per milliliter of culture medium. In other words, the cell density of the pluripotent stem cells that are differentiated towards the preselected cell type in the method as disclosed herein is preferably between 1×10⁴-1×10⁶ cells per milliliter of culture medium, preferably between 5×10⁴-5×10⁵ cells per milliliter of culture medium.

In a preferred embodiment of the method as disclosed herein, the cells in step b) are introduced in the form of cell aggregates, and preferably the cell aggregates have a size of between 10-150 micrometer, preferably between 25-140 micrometer, preferably selected from the group consisting of between 20-80 micrometer, 30-60 micrometer, 90-140 micrometer, and 100-120 micrometer.

In a preferred embodiment of the method as disclosed herein, and wherein the cells in step b) are introduced in the form of a single cell suspension, the single cells are allowed to form aggregates, e.g. during step c), wherein the size of the aggregates that are formed are between 10-150 micrometer, preferably between 25-140 micrometer, preferably selected from the group consisting of between 20-80 micrometer, 30-60 micrometer, 90-140 micrometer, and 100-120 micrometer, preferably about 24-72 hours after introduction of the cell suspension with single cells and/or commencement of the culturing of the cells/mixing of the culture medium.

Therefor also provided is for a method according to the invention, wherein, when the cells in step b) are introduced in the form of a single cell suspension and wherein the cells are allowed to form aggregates having a size of between 10-150 micrometer, preferably between 25-140 micrometer, preferably selected from the group consisting of between 20-80 micrometer, 30-60 micrometer, 90-140 micrometer, and 100-120 micrometer.

It was found that cell aggregates of this size allow for optimal yield and quality of the preselected cell type with the method as disclosed herein, in particular in case the cells in step b) are introduced in the form of a single cell suspension

In a preferred embodiment of the method as disclosed herein the amount of preselected cell type manufactured is at least 10 times the amount of pluripotent stem cells introduced in step b), preferably at least 15 times, at least 20 times, or at least 25 times, preferably between 10-100 times, between 15-80 times or between 20-75 times. The above embodiment in particular relates to the preselected cell type that is collected from the cell aggregates that are collected in step j). Therefore, in some embodiments or aspects there is provided for a method for the in vitro manufacture of at least 10 times the amount of pluripotent stem cells used of a preselected cell type differentiated from said amount of pluripotent stem cells, preferably in a closed culture system, wherein the method comprises the steps as disclosed above and herein. Preferably, the method is for preselected cell types in the form of cell aggregates.

In those embodiments wherein the preselected cell type may be collected as single cells in the culture medium, for example is case of cells of the hematopoietic linage, the amount of preselected cell type manufactured is preferably at least 10-1000 times, 20-1000 times, preferably 50-1000 times the amount of pluripotent stem cells introduced in step b), for example at least 20 times, 30 times, 50 times, 80 times, 100 times, 250 times or more. Therefore, in some embodiments or aspects there is provided for a method for the in vitro manufacture of at least 50 times the amount of pluripotent stem cells used of a preselected cell type differentiated from said amount of pluripotent stem cells, preferably in a closed culture system, wherein the method comprises the steps as disclosed above and herein. Preferably, the method is for preselected cell types in the form of single cells.

In some embodiments, the amount of preselected cell type manufactured is at least the above mentioned number(s) of times the amount of pluripotent stem cells that are induced to proliferate towards the preselected cell type in step b) or in step g).

In a preferred embodiment of the method as disclosed herein the cell aggregates collected in step j) have a size of less than 1000 micrometer, preferably have a size of between 10-1000 micrometer, 20-750 micrometer or 50-500 micrometer. In this embodiment the aggregates have a diameter of less than 1 mm, preferably having a diameter within the range of 10 to 1000 μm, preferably 50 μm to 500 μm. Aggregate size is deemed relevant for two reasons. First it was found that the aggregate size at the start of proliferation and/or differentiation may impact differentiation efficiency. Secondly, during the differentiation process the amount of cells should increase as reflected in larger aggregates. Once aggregates become too large nutrient availability becomes a rate limiting step resulting in necrotic cores and suboptimal bioprocess yields. In addition, it is believed that too large aggregates provide for less homogenous populations of pre-selected cells types, possibly due to local effects within the aggregate. In one aspect of the method as disclosed herein the aggregate size can optimized using modulation of spinning speed/agitation during the aggregate formation period, e.g. the first 24 h after inoculation of single cells.

In a preferred embodiment of the method as disclosed herein the volume of the culture medium in the culture vessel is at least 1 liter, preferably at least 2 liter, 3 liter, 4 liter, 5 liter, 6 liter, 7.5 liter, or 10 liter, preferably wherein the culture medium in the culture vessel is between 1 liter and 100 liter, preferably between 5 liter and 50 liter.

The present method now allows for the manufacture of the preselected cell types in, preferably closed, culture systems having increased volumes (and thereby supporting high yields) of, for example 3, 10, 40 or even 100 liter. This is a major advantage of the method as disclosed herein.

In a preferred embodiment of the method as disclosed herein at least 1×10⁶ cells/ml culture medium is manufactured, preferably at least 1.5×10⁶ cells/ml, 2.0×10⁶ cells/ml, 3.0×10⁶ cells/ml, or 5.0×10⁶ cells/ml, for example at least 1.5×10⁶ cells/ml, 2.0×10⁶ cells/ml, 3.0×10⁶ cells/ml, 4.0×10⁶ cells/ml, 5.0×10⁶ cells/ml, 8.0×10⁶ cells/ml, 12.0×10⁶ cells/ml or 20.0×10⁶ cells/ml.

The method as disclosed herein was found to be so robust that the method can be continued until at least 1×10⁶ cells/ml culture medium is manufactured, preferably at least 1.5×10⁶ cells/ml, 2.0×10⁶ cells/ml, 3.0×10⁶ cells/ml, or 5.0×10⁶ cells/ml, for example at least 1.5×10⁶ cells/ml, 2.0×10⁶ cells/ml, 3.0×10⁶ cells/ml, 4.0×10⁶ cells/ml, 5.0×10⁶ cells/ml, 8.0×10⁶ cells/ml, 12.0×10⁶ cells/ml or 20.0×10⁶ cells/ml.

Thus in a preferred embodiment the method comprises that it is continued until at least such number of cells are manufactured. By producing such number of cells the method provides for the long felt need to be able to produce, in one system, high number of cells of a preselected cell type and that may be used in cellular therapy.

In connection with the above, in a preferred embodiment of the method as disclosed herein at least 1×10⁹ preselected cells are manufactured, preferably at least 10×10⁹, 25×10⁹, 100×10⁹, 200×10⁹, or 500×10⁹ preselected cells are manufactured. Again, the method as disclosed herein was found to be so robust that the method can be continued until such total number of cells of the preselected cell type are produced (e.g. at least 1×10⁹ preselected cells). Thus in a preferred embodiment the method comprises that it is continued until at least such number of cells are manufactured. By producing such number of cells the method again provides for the long felt need to be able to produce, in one system, high number of cells of a preselected cell type and that may be used in cellular therapy.

In a preferred embodiment of the method as disclosed herein optional step f) is omitted and/or in step g)-i) are repeated at least once, preferably at least 2, 3, 4, 5, 6, 7, 8, 9, 10, 15, 20, 25 times.

Preferably step f) is omitted in the method as disclosed herein. Even more preferably in step b) of the method as disclosed herein a culture medium for inducing differentiation of the pluripotent stem cells towards the preselected cell type is used, therewith omitting any step of proliferation of the pluripotent stem cells. In other words, in step b) the number or density per volume culture medium allows for omitting any proliferation of the pluripotent stem cells, and allows for immediately inducing of the pluripotent stem cells towards the preselected cell type.

Preferably step g)-i) are repeated at least once, preferably at least 2, 3, 4, 5, 6, 7, 8, 9, 10, 15, 20, 25 times. As the skilled person understands, repeating steps g)-i) includes repeating these steps using a culture medium that is the same or different from the previous used culture medium, as long as the culture medium allows for the further differentiation (which, throughout the whole application, is also to be understood to include culture media that maintain the differentiated state of the cells once the cells have differentiated into the preselected cell type) of the cells. For example, subsequent culture media may differ only in the compounds that are included and that steer, induce, enhance and/or maintain differentiation or state of differentiation of the cells, or may differ with respect to other ingredients (e.g. glucose) or both.

It was found that the method as disclosed herein allow for repeating steps g)-i) several times. This is in particular advantageous in cases wherein the differentiation of the cells towards the preselected cell types requires different steps or different culture medium compositions in order to obtain the preselected cell type.

In a preferred embodiment of the method as disclosed herein step c) or h) is, each independently, for at least 12 hours, 1, 2, 3, 4, 5, 6, or 7 days, preferably no more than 10, preferably 7 days and/or wherein step h) is for at least 12 hours, 1, 2, 3, 4, 5, 6, or 7 days.

In steps c) and or h) the cells are cultured in the presence of a culture medium, in the following steps, the culture medium is or may be replaced by a subsequent culture medium. Replacement of the culture medium may be at the intervals described above (i.e. after 12 hours, 1, 2, 3, 4, 5, 6, or 7 days, preferably no more than 10, preferably 7 days of cultivating in the culture medium during step c) or h)). In other words, the cells are preferably contacted with a culture medium, before it is partially replaced in the subsequent step of the method as disclosed herein, for at least once every 12 hours, 1, 2, 3, 4, 5, 6, or 7 days, preferably no more than 10, preferably 7 days.

In a preferred embodiment of the method as disclosed herein the method (steps a)-j)) is performed over a period of time of at least 5, 6, 7, 10, 11, 12, 13, 14, 15, 16, 17, 18, 19, 20 days, preferably between 7 and 90 days, 10-60 days, 15-40 days, or 15-30 days. By performing the method for the provided number of days, the method provides for high yield of the preselected cell types.

In view of the above, and only by way of illustration, and in the context of the method as disclosed herein, manufacturing of preselected cell types may, for example, be performed as follows:
Situation A: proliferating pluripotent stem cells for a period of 2 days in a first proliferation medium, collecting the first proliferation medium and providing a second proliferation medium (with the same or different composition), continue proliferation of the pluripotent stem cells for a period of 1 day, collecting the second proliferation medium and providing a first differentiation medium, differentiating the cells for a period of 1 day, collecting the first differentiation medium and providing a second differentiation medium (with the same or different composition), continue differentiation of the cells for a period of 1, 2, 3, or 4 days, collecting the second differentiation medium and providing a subsequent differentiation medium (with the same or different composition), continue differentiation of the cells for a period of 1, 2, 3, or 4 days, collecting the subsequent differentiation medium and providing a next subsequent differentiation medium (with the same or different composition), and repeating the collection of the differentiation medium, the providing of the next differentiation medium (with the same or different composition) and the continuation of the differentiation of the cells until or as long as the preselected cell types can be obtained.
Situation B: providing a first differentiation medium to the pluripotent stem cells, differentiating the cells for a period of 2 days, collecting the first differentiation medium and providing a second differentiation medium (with the same or different composition), continue differentiation of the cells for a period of, for example 1, 2, 3, or 4 days, collecting the second differentiation medium and providing a subsequent differentiation medium (for example with the same composition), continue differentiation of the cells for a period of 1, 2, 3, or 4 days, collecting the subsequent differentiation medium and providing a next subsequent differentiation medium (for example, again with the same composition, but with a different pH), and repeating the collection of the differentiation medium, the providing of the next differentiation medium (with the same or different composition) and the continuation of the differentiation of the cells until or as long as the preselected cell types can be obtained.

As illustrated herein, in a preferred embodiment there is provided for the method as disclosed herein, wherein the compositions of the different culture media for proliferation of the pluripotent stem cells are the same or different and/or wherein the compositions of the different culture media for inducing differentiation of the (pluripotent stem) cells towards the preselected cell type are the same or different.

By providing culture media for proliferation or differentiation that are different, it is possible to provide multiple differentiation cycles or to combine a proliferation step followed by one or more differentiation steps in the same culture vessel.

In a preferred embodiment, there is provided for the method as disclosed herein wherein the culture medium provided in step b) or step g) comprises a Rho-associated protein kinase inhibitor, such as, for example Y-27632 dihydrochloride or Fasudil.

In another embodiment, and as already discussed above, there is provided for the method as disclosed herein wherein in step e) and/or j) at most 95 vol. %, 90 vol. %, 85 vol. %, or 80 vol. % of the culture medium in the culture vessel is collected.

At the same time it was found that, preferably, at least 50 vol. %, 60 vol. % or 70 vol. % of the culture medium is collected from the culture vessel. Therefore, in some embodiments between, for example 50-95 vol. %, or between 60-90 vol. % or between 70-90 vol. % of the culture medium is collected from the culture vessel.

Preferably the amount of culture medium that is removed from the culture vessel is as such that the ratio of culture medium that remains behind in the culture vessel and the fresh culture medium that is added to the culture vessel in case a new, fresh culture medium is provided to the culture system is from 1:1 to 1:15, for example 1:3-1:10, for example 1:4-1:8.

In another embodiment there is provided for the method according to the invention, wherein the preselected cell type is a is a cardiovascular cell, a cardiomyocyte, an endothelial cell, a cell of the hematopoietic linage, a hematopoietic progenitor cell, a cell differentiated from a hematopoietic progenitor cell, a monocyte, a macrophage, a T-cell, a B-cell, a NK-cell, a dendritic cell, a neuronal cell, a retinal cell, a lung cell, a liver cell, or a pancreatic cell.

Although the method as disclosed herein is not in particular limited to the differentiation towards a specific (differentiated) cell type, it was found that the current method is in particular suitable to obtain the above-mentioned preselected cell types in the form of aggregates and/or from the culture medium in the form of single cells (for example secreted from the cell aggregates).

The skilled person understands that by preselecting a particular cell type towards which the pluripotent stem cells will be differentiated, using the method as disclosed herein, this also determines the types of culture medium (and compounds required) that are suitable for use in said method as disclosed herein.

As discussed before, it was surprisingly found that the method as disclosed herein allows for the differentiation towards a wide variety of cell types by making use of various known differentiation protocols, albeit adjusted in line with the method as disclosed herein. For example, the method can be used wherein the preselected cell is a cardiovascular cell, a cardiomyocyte, an endothelial cell, a cell of the hematopoietic linage, a hematopoietic progenitor cell, a cell differentiated from a hematopoietic progenitor cell, a monocyte, a macrophage, a T-cell, a B-cell, a NK-cell, a dendritic cell, a neuronal cell, a retinal cell, a lung cell, a liver cell, or a pancreatic cell.

In a further embodiment there is provided for the method as disclosed herein wherein the part of the culture medium that is collected or the culture medium that is collected comprises single cells and/or non-aggregated cells, preferably wherein these single cells of non-aggregated cells are selected from the group consisting of a cell of the hematopoietic lineage, a hematopoietic progenitor cell, a cell differentiated from a hematopoietic progenitor cell, a monocyte, a macrophage, a T-cell, a B-cell, a NK-cell, or a dendritic cell. The method as disclosed herein is also in particular suitable for cultures that contain both aggregates and single cells (as preselected cell type to obtain) as seen in hematopoietic differentiation processes. In such cultures, the aggregates continue to secrete/shed hematopoietic cells which need to be harvested as single cells from the culture medium at a regular interval. The current method allows without filtration means and/or without centrifugation means to collect such cells from the collected culture medium during the different step of the method.

In a further preferred embodiment there is provided for the method as disclosed herein wherein the culture medium for inducing differentiation of the pluripotent stem cells towards the preselected cell type proliferation and that is introduced into the culture vessel comprises one or more compounds that induce differentiation of the pluripotent stem cells towards the preselected cell type by inhibiting or activating certain signaling pathways required for early development.

The culture media used in the methods of the present invention, in particular the culture media used to differentiate can, for example, comprise a signaling activator or a signaling inhibitor. The term “activator”, as used herein, is defined as a compound/molecule enhancing or achieving the activity of a target molecule and/or signaling pathway and that, for example, promotes differentiation of the cells towards the preselected cell type. The term “activator” encompasses both molecules/compounds that have a directly activating effect on the specific signaling pathway but also molecules that are indirectly activating, e.g. by interacting for example with molecules that negatively regulate (e.g. suppress) said pathway. The activator can also be an agonist of the signaling pathway (receptor) to be activated.

The compound/molecule that can be used as an activator can be any compound/molecule, which can activate the respective pathway or which inhibits a suppressor of the pathway to be activated.

An activator may enhance or increase the pathway to be activated by 10%, 20%, 30%, 40%, 50%, 60%, 70%, 80%, 90%, 100% or more when compared to the activity of the pathway without the addition (or before the addition) of the activator.

On the contrary to the activator or signaling/pathway activator as described herein an “inhibitor” as used herein is defined as a compound/molecule reducing or blocking the activity of a target molecule and/or signaling pathway. The term “inhibitor” encompasses both molecules/compounds that have a directly reducing/blocking effect on the specific signaling pathway but also molecules that are indirectly inhibiting, e.g. by interacting for example with molecules that positively regulate (e.g. activate) said pathway. The inhibitor can also be an antagonist of the pathway (receptor) to be inhibited.

The compound/molecule that can be used as an inhibitor can be any compound/molecule, which can reduce or block the respective pathway or which inhibits an activator of the signaling (pathway) to be inhibited. Exemplary inhibitors can include suitable binding proteins as described herein, which are directed e.g. against activators of a certain pathway.

An inhibitor may reduce or decrease the pathway to be inhibited by 10%, 20%, 30%, 40%, 50%, 60%, 70%, 80%, 90% or more when compared to the activity of the pathway without the addition of the inhibitor. A block of the pathway to be inhibited is present when the pathway is inhibited by 100% when compared to the activity of the pathway without the addition (or before the addition) of the inhibitor.

The media as used in the methods of the present invention can for example comprise an activin/TGF-β inhibitor. The activin/TGF-β signaling pathway is known in the art and for example described in Heldin, Miyazono and ten Dijke (1997) “TGF-bold beta signaling from cell membrane to nucleus through SMAD proteins.” Nature 390, 465-471. In short, Receptor ligands, including, for example, TGFB1, TGFB2, TGFB3, ACTIVIN A, ACTIVIN B, ACTIVIN AB and/or NODAL, bind to a heterotetrameric receptor complex comprising two type I receptor kinases, including, for example, TGFBR2, ACVR2A, and/or ACVR2B, and two type II receptor kinases, including, for example, TGFBR1 (ALK5), ACVR1 B (ALK4) and/or ACVR1 C (ALK7). This binding triggers phosphorylation and activation of a heteromeric complex consisting of an R-smad, including, for example, SMAD2, and/or SMAD3, and a Co-smad, including, for example, SMAD4. Accordingly, the term “activator of the activin/TGF-β signaling pathway” refers to an activator of any one of the above recited molecules that form part of this signaling pathway, while the term “inhibitor of the activin/TGF-β signaling pathway” refers to inhibitors of any one of the above recited molecules that form part of this signaling pathway. In addition, such an activator can be an agonist of the ACVR2A and/or ACVR1 B (ALK4) receptor or an agonist of the TGF RII receptor and/or ALK5 receptor. Such an inhibitor can be an antagonist of the ACVR2A and/or ACVR1 B (ALK4) receptor or an antagonist of the TGF RII receptor and/or ALK5 receptor. In principle such inhibitors/activators of the activin/TGF-β signaling pathway are known to the person skilled in the art and are commercially available.

The invention contemplates that the activin/TGF-β inhibitor is an inhibitor of the TGF-β type I receptor activin receptor-like kinase(s). Further envisioned by the present invention is that the activin/TGF-β inhibitor inhibits ALK5, ALK4 and/or ALK7. Exemplary but non-limiting examples of an activin/TGF-β inhibitor are A-83-01 (3-(6-Methyl-2-pyridinyl)-N-phenyl-4-(4-quinolinyl)-1H-pyrazole-1-carbothioamide; CAS No.: 909910-43-6), D4476 (4-[4-(2,3-Dihydro-1,4-benzodioxin-6-yl)-5-(2-pyridinyl)-1H-imidazol-2-yl]benzamide; CAS No.: 301836-43-1), GW788388 (4-[4-[3-(2-Pyridinyl)-1H-pyrazol-4-yl]-2-pyridinyl]-N-(tetrahydro-2H-pyran-4-yl)-benzamide; CAS No.: 452342-67-5), LY364947 (4-[3-(2-pyridinyl)-1H-pyrazol-4-yl]-quinoline; CAS No.: 396129-53-6), R268712 (4-[2-Fluoro-5-[3-(6-methyl-2-pyridinyl)-1H-pyrazol-4-yl]phenyl]-1H-pyrazole-1-ethanol; CAS No.: 879487-87-3), SB-431542 (4-(5-Benzol[1,3]dioxol-5-yl-4-pyrldin-2-yl-1H-imidazol-2-yl)-benzamide hydrate; CAS No.: CAS Number 301836-41-9), SB-505124 (2-(5-Benzo[1,3]dioxol-5-yl-2-tert-butyl-3H-imidazol-4-yl)-6-methylpyridine hydrochloride hydrate; CAS No.: 694433-59-5), SD208 (2-(5-Chloro-2-fluorophenyl)\hat{ }-[(4-pyridyl)amino]pteridine; CAS No.: 627536-09-8), SB-525334 (6-[2-tert-Butyl-5-(6-methyl-pyridin-2-yl)-1H-imidazol-4-yl]-quinoxaline; CAS No.: 356559-20-1) and ALK5 Inhibitor II (CAS: 446859-33-2). The activin/TGF-β inhibitor can thus be SB-431542.

The activin/TGF-β inhibitor such as SB-431542 can be employed in a concentration of between about 0.01 μM and about 1 M, more preferably between about 5 μM and about 15 μM, and most preferably the amount is about 10 μM. For example, SB-431542 can be obtained from Ascent Scientific.

The canonical Wnt signaling pathway is known to the person skilled in the art and for example described in Logan and Nusse (Annu. Rev. Cell Dev. Biol. (2004) 20:781-810). In short, a Wnt ligand binds to Frizzled receptors, which triggers displacement of the multifunctional kinase GSK-3P from a regulatory APC/Axin/GSK-3p-complex. In the absence of Wnt-signal (Off-state), β-catenin, is targeted by coordinated phosphorylation by CK1 and the APC/Axin/GSK-3p-complex leading to its ubiquitination and proteasomal degradation through the β-TrCP/SKP pathway. In the presence of Wnt ligand (On-state), the co-receptor LRP5/6 is brought in complex with Wnt-bound Frizzled. This leads to activation of Dishevelled (Dvl), which displaces GSK-3P from APC/Axin. The transcriptional effects of Wnt ligand is mediated via Rac1-dependent nuclear translocation of β-catenin and the subsequent recruitment of LEF/TCF DNA-binding factors as co-activators for transcription. Exemplary Wnt ligands include for example Wnt1, Wnt3, Wnt3a, Wnt4, Wnt5a, Wnt7a, Wnt7b, and/or Wnt11

Accordingly, the term “canonical WNT-signaling activator” as described herein refers to an activator of any one of the above recited molecules that form part of this signaling pathway. Exemplary canonical WNT-signaling activators include Norrin, R-spondin 2 or WNT protein. However, the canonical WNT-signaling activator can also block Axin or APC. This can be achieved for example via siRNA or miRNA technology. It is also encompassed by the present invention that the canonical WNT-signaling activator is a GSK-3 inhibitor. Exemplary GSK-3 inhibitors include CHIR 99021 (6-[[2-[[4-(2,4-Dichlorophenyl)-5-(5-methyl-1H-imidazol-2-yl)-2 pyrimidinyl]amino]ethyl]amino]-3-pyridinecarbonitrile; CAS No.: 252917-06-9), SB-216763 (3-(2,4-Dichlorophenyl)-4-(1-methyl-1H-indol-3-yl)-1H-pyrrole-2,5-dione; CAS No.: 280744-09-4), 6-bromoindirubin-3′-oxime (CAS No.: CAS 667463-62-9), Tideglusib (4-Benzyl-2-(naphthalen-1-yl)-1,2,4-thiadiazolidine-3,5-dione), GSK-3 inhibitor 1 (CAS No.: 603272-51-1), AZD1080 (CAS No.: 612487-72-6), TDZD-8 (4-Benzyl-2-methyl-1,2,4-thiadiazolidine-3,5-dione; CAS No.: 327036-89-5), TWS1 19 (3-[[6-(3-aminophenyl)-7H-pyrrolo[2,3-d]pyrimidin-4-yl]oxy]-phenol; CAS No.: 601514-19-6), CHIR-99021 (CAS No.: 252917-06-9), CHIR-98014 (N6-[2-[[4-(2,4-dichlorophenyl)-5-(1H-imidazol-1-yl)-2-pyrimidinyl]amino]ethyl]-3-nitro-2,6-Pyridinediamine; CAS No.: 252935-94-7), SB 415286 (3-[(3-Chloro-4-hydroxyphenyl)-amino]-4-(2-nitrophenyl)-1H-pyrrol-2,5-dione; CAS No.: 264218-23-7), LY2090314 (3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; CAS No.: 603288-22-8), AR-A014418 (N-(4-Methoxybenzyl)-N′-(5-nitro-1,3-thiazol-2-yl)urea; CAS No.: 487021-52-3 and/or IM-12 (3-(4-Fluorophenylethylamino)-1-methyl-4-(2-methyl-1H-indol-3-yl)-1H-pyrrole-2,5-dione; CAS No.: 1 129669-05-1). Thus, the GSK-3 inhibitor can also be CHIR 99021.

The canonical WNT-signaling activator such as CHIR 99021 can be employed in a concentration of between about 0.01 μM and about 1 M, more preferably between about 0.1 μM and about 10 μM, or about 0.1 μM and about 5 μM, more preferable between 2 and 8 μM, and most preferably the amount is between 4 and 6 μM. CHIR 99021 can for example be obtained from Axon Medchem.

Accordingly, the term “canonical WNT-signaling inhibitor” as described herein refers to an activator of any one of the above recited molecules that form part of this signaling pathway. Exemplary canonical WNT-signaling inhibitors include IWP-2, IWP-3, IWP-4, IWP-L6, XAV939 and IWR-1-ENDO. The canonical WNT-signaling inhibitors such as IWPL-6 and XAV can be employed in a concentration of between about 0.01 μM and about 1 M, more preferably between about 0.1 μM and about 10 μM or between about 0.1 μM and 5 μM, and most preferably the amount is between 1 and 10 μM for XAV939, for example between 2 and 8 μM, or 3-7 μM, and between 0.1 μM and 1 μM, for example between 0.15 μM and 0.50 μM or between 0.20 μM and 0.30 μM for IWP-L6.

The media as used in the methods of the present invention can additionally or alternatively comprise a BMP signaling inhibitor. The BMP signaling pathway is known to the person skilled in the art and for example described in Jiwang Zhanga, Linheng Lia (2005) BMP signaling and stem cell regulation Developmental Biology Volume 284, Issue 1, 1 Aug. 2005, Pages 1-11.

In short, BMP functions through receptor-mediated intracellular signaling and subsequently influences target gene transcription. Two types of receptors are required in this process, which are referred to as type I and type II. While there is only one type II BMP receptor (BmprII), there are three type I receptors: Alk2, Alk3 (BmprI a), and Alk6 (BmprI b). BMP signal transduction can take place over at least two signaling pathways. The canonical BMP pathway is mediated by receptor I mediated phosphorylation of SmadI, Smad5, or Smad8 (R-Smad). Two phosphorylated R-Smads form a heterotrimeric complex coaggregate with a common Smad4 (co-Smad). The Smad heterotrimeric complex can translocate into the nucleus and can cooperate with other transcription factors to modulate target gene expression. A parallel pathway for the BMP signal is mediated by TGFpi activated tyrosine kinase 1 (TAK1, a MAPKKK) and through mitogen activated protein kinase (MAPK), which also involves cross-talk between the BMP and Wnt pathways. The inhibitors of BMP signaling can only block/reduce the canonical BMP pathway. Thus, the BMP signaling inhibitor can be a canonical BMP signaling inhibitor. One such inhibitor selective for canonical BMP signaling pathway is dorsomorphin. Exemplary, but non-limiting, examples of BMP signaling inhibitors include chordin, noggin, DMH1 (CAS 120671 1-16-1), K 02288 (3-[(6-Amino-5-(3,4,5-trimethoxyphenyl)-3-pyridinyl]phenol; CAS No.: 1431985-92-0), dorsomorphin (6-[4-(2-Piperidin-1-ylethoxy)phenyl]-3-pyridin-4-ylpyrazolo[1,5-a]pyrimidine; CAS No.: 866405-64-3) and LDN 193189 (4-[6-[4-(1-Piperazinyl)phenyl]pyrazolo[1,5-a]pyrimidin-3-yl]-quinoline hydrochloride, CAS No.: 1062368-24-4). The BMP signaling inhibitor can also be dorsomorphin.

The media as used in the methods of the present invention can additionally or alternatively comprise a SHH-pathway activator. The “Hedgehog signaling pathway” or “SHH pathway” is well known in the art and has been described, for example, in Choudhry et al. (2014) “Sonic hedgehog signaling pathway: a complex network.” Ann Neurosci. 21 (1):28-31. Hedgehog ligands, including, for example, Sonic hedgehog, Indian hedgehog, and/or Desert hedgehog, bind to the receptor, including, for example, Patched or the patched—smoothened receptor complex, which induces a downstream signaling cascade. Downstream target genes of SHH signaling include GLI1, GLI2 and/or GLI3. Accordingly, the term “activator of the Hedgehog signaling pathway” also refers to an activator of any one of the above recited molecules that form part of this signaling pathway.

Exemplary activators of the Hedgehog signaling (SHH) include purmorphamine (PMA; 2-(1-Naphthoxy)-6-(4-morpholinoanilino)-9-cyclohexylpurine 9-Cyclohexyl-N-[4-(4-morpholinyl)phenyl]-2-(1-naphthalenyloxy); CAS No.: 483367-10-8), SHH, smoothened agonist (SAG; 3-chloro-N-[trans-4-(methylamino)cyclohexyl]-N-[[3-(4-pyridinyl)phenyl]methyl]-benzo[b]thiophene-2-carboxamide; CAS No.: 912545-86-9) and Hh-Ag 1 0.5 (3-chloro-4,7-difluoro-N-(4-(methylamino)cyclohexyl)-N-(3-(pyridin-4-yl)benzyl)benzo[b]thiophene-2-carboxamide; CAS No.: 612542-14-0) as well as Gli-2. The SHH-pathway activator can also be selected from the group consisting of purmorphamine, SHH, SAG Analog and Gli-2. The SHH-pathway activator can therefore be purmorphamine. The SHH pathway activator can also be a recombinant or truncated form of SHH, which retains SHH pathway activating functions such as e.g. SHH C24II.

The SHH signaling pathway activator such as purmorphamine can be employed in a concentration of between about 0.25 μM and about 1 M, more preferably between about 0.4 μM and about 0.5 μM, and most preferably the amount is about 0.5 μM. The SHH signaling pathway activator such as SHH can also be employed between about 50 and about 1000 ng/ml. The SHH signaling pathway activator such as SHH C24II can also be employed in a concentration of about 10 and about 500 ng/ml. The SHH signaling pathway activator such as SAG can be employed in a concentration of about 1 and about 100 nM. The SHH signaling pathway activator such as Hh-Ag1 0.5 can also be employed in a concentration of about 1 and about 50 nM.

The media as used in the methods of the present invention can additionally or alternatively comprise a protein or steroid hormone growth factor selected from the group of Adrenomedullin (AM), Angiopoietin (Ang), Autocrine motility factor, Ciliary neurotrophic factor (CNTF), Leukemia inhibitory factor (LIF), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1(FGF1), Fibroblast growth factor 2(FGF2), Fibroblast growth factor 3(FGF3), Fibroblast growth factor 4(FGF4), Fibroblast growth factor 5(FGF5), Fibroblast growth factor 6(FGF6), Fibroblast growth factor 7(FGF7), Fibroblast growth factor 8(FGF8), Fibroblast growth factor 9(FGF9), Fibroblast growth factor 10(FGF10), Fibroblast growth factor 11(FGF11), Fibroblast growth factor 12(FGF12), Fibroblast growth factor 13(FGF13), Fibroblast growth factor 14(FGF14), Fibroblast growth factor 15(FGF15), Fibroblast growth factor 16(FGF16), Fibroblast growth factor 17(FGF17), Fibroblast growth factor 18(FGF18), Fibroblast growth factor 19(FGF19), Fibroblast growth factor 20(FGF20), Fibroblast growth factor 21(FGF21), Fibroblast growth factor 22(FGF22), Fibroblast growth factor 23(FGF23), Foetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL4, IL-5, IL6, IL7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO),

Transforming growth factor alpha (TGF-α), Transforming growth factor beta (TGF-β), Tumor necrosis factor-alpha (TNF-α), Vascular endothelial growth factor (VEGF).

The above signaling molecules and pathways, and the role that inhibitors and/or activator of that pathways play in differentiation towards various cell types are well-known to the skilled person. The skilled person also knows that, in order to differentiate towards a particular preselected cell type, a combination of the above mentioned activator and inhibitors may be used, either in the same culture medium or is consecutive culture media. For example, one may use an inhibitor of a particular pathway in a first differentiation medium, followed by an activator of the same pathway in a second or next differentiation medium.

In a further embodiment of the method as disclosed herein the culture medium provided in step b) and/or g), preferably step g) comprises polyvinyl alcohol, preferably in a range of about 0.1-10 mg/ml culture medium. It was found that the presence of PVA in the culture medium is desirable in the method of the invention,

In a further embodiment of the method as disclosed there is provided that the culture medium for inducing differentiation of the cells towards the preselected cell type comprises one or more compounds that induce differentiation of the cells towards the preselected cell type, preferably wherein the one or more compounds are selected from the group consisting of Wnt-pathway activators, Wnt-pathway inhibitors, Activin-pathway activators, TGFβ-pathway activators, BMP-pathway activators, Activin-pathway inhibitors, TGFβ-pathway inhibitors, BMP-pathway inhibitors, and VEGF-pathway activators.

In another embodiment there is provided for the method as disclosed herein wherein the one or more compounds that induce the differentiation of the pluripotent stem cells towards the preselected cell type is/are selected from the group consisting of compounds that induce the differentiation of the pluripotent stem cells to a cardiovascular cell, a cardiomyocyte, an endothelial cell, a cell of the hematopoietic linage, a hematopoietic progenitor cell, a cell differentiated from a hematopoietic progenitor cell, a monocyte, a macrophage, a T-cell, a B-cell, a NK-cell, a dendritic cell, a neuronal cell, a retinal cell, a lung cell, a liver cell, or a pancreatic cell.

In another embodiment there is provided for the method as disclosed herein wherein the culture media for inducing differentiation of the pluripotent stem cells towards the preselected cell type comprises a thyroid hormone and/or thyroid hormone analog. The terms ‘thyroid hormone’ or ‘thyroid hormone analogs’ as used herein refers to thyroid hormone (also referred to as triiodothyronine (T3)) as well as to T4 and to other compounds which are analogue to the thyroid hormone T3 or mimic thyroid hormone T3's actions. Non-limiting examples include thyroid hormone receptor agonist compounds such as DITPA (also referred to as 3,5-diiodothyroproprionic acid or DITPA), GC-1 compounds (which is a thyroid hormone receptor subtype beta (TRbeta) selective agonist from Bristol-Myers Squibb), RO compounds (which is a thyroid hormone receptor subtype beta 1 (TRbeta) selective agonist from Roche Pharmaceuticals), C023 compound (which is a thyroid hormone subtype alpha 1 (TRalphal) selective agonist from KaroBio), and KB21 15 (which is a thyroid hormone receptor subtype beta (THbeta) selective agonist from KaroBio).

In another embodiment, the culture medium for differentiation comprises glucose and/or galactose. In another embodiment the culture medium does not comprise glucose and/or galactose. In some embodiments, the culture medium comprises serum, in other embodiments the culture medium is serum free.

In another embodiment, the culture medium for differentiation is chemically defined. In another embodiment all components are cGMP compliant.

In another embodiment the culture medium is specifically optimized for the use in bioreactors, e.g. to reduce shear stress and foaming. Reagents that are particularly useful for this purpose are polyvinyl alcohol and Pluronic F68.

In another embodiment there is provided for the method as disclosed herein wherein the pluripotent stem cells is an induced pluripotent stem cell, preferably a human pluripotent stem cell, or a human induced pluripotent stem cell.

In another embodiment there is provided for the method as disclosed herein wherein the cell differentiated towards the preselected cell type are obtained from the cell aggregate or from the collected culture medium.

In another embodiment cells of the hematopoietic lineage such as hematopoietic stem cells, hematopoietic progenitor cells, monocytes, macrophages, T-cells, NK-cells, B-cells and or dendritic cells are isolated as single cells from the culture medium.

In another embodiment, secreted proteins and or secreted exosomes are isolated from the culture medium. It is believed that exosomes that, for example, arise in a cardiac differentiation process are particularly useful for the treatment of cardiac diseases, that exosomes that arise during a liver differentiation process are particularly useful for the treatment of liver diseases etc.

In some embodiments, the preselected cell types obtained with the method as disclosed herein can be used in cell therapy. The cell therapy can be a form of regenerative medicine wherein cells are transplanted to restore organ function or are an immune therapy e.g. to treat cancer.

In some embodiments, the preselected cell type obtained with the method as disclosed herein can be formulated in pharmaceutically acceptable amounts and in pharmaceutically acceptable compositions. Such compositions may, in some embodiments, contain salts, buffering agents, preservatives, and optionally other therapeutic agents. Pharmaceutical compositions also may contain, in some embodiments, suitable preservatives. The compositions disclosed herein have numerous therapeutic utilities, including, e.g., organ repair, the treatment of cancers, autoimmune diseases and infectious diseases.

Therefor there is also provided for a pharmaceutical composition for use in cell therapy or a method of treatment by cell therapy wherein the cell therapy comprises the step of providing a preselected cell type to a subject in need thereof and wherein the preselected cell type has been manufactured with a method as defined and disclosed herein or wherein the cell therapy comprises the step of manufacture of a preselected cell type with a method as defined or disclosed herein and providing the preselected cell type to a subject in need thereof.

There is also provided for the use of a closed culture system in differentiating pluripotent stem cells towards a preselected cell type, preferably according to a method as disclosed herein.

Finally there is provided for a, preferably closed, culture system at least 1×10⁹ preselected cells, preferably at least 10×10⁹, 25×10⁹, 100×10⁹, 200×10⁹, or 500×10⁹ preselected cells and/or comprising at least 1 liter, preferably at least 2 liter, 3 liter, 4 liter, 5 liter, 6 liter, 7.5 liter, or 10 liter culture medium, preferably between 1 liter and 100 liter, preferably between 5 liter and 50 liter culture medium comprising at least 1×10⁶ cells/ml culture medium, preferably at least 1.5×10⁶ cells/ml, 2.0×10⁶ cells/ml, 3.0×10⁶ cells/ml, or 5.0×10⁶ cells/ml, for example at least 1.5×10⁶ cells/ml, 2.0×10⁶ cells/ml, 3.0×10⁶ cells/ml, 4.0×10⁶ cells/ml, 5.0×10⁶ cells/ml, 8.0×10⁶ cells/ml, 12.0×10⁶ cells/ml or 20.0×10⁶ cells/ml.

It will be understood that all details, embodiments and preferences discussed with respect to one aspect of embodiment as disclosed herein is likewise applicable to any other aspect or embodiment as disclosed herein and that there is therefore not need to detail all such details, embodiments and preferences for all aspect separately.

Having now generally described the invention, the same will be more readily understood through reference to the following examples which is provided by way of illustration and is not intended to be limiting of the present invention.

### EXAMPLES

### Example 1 Large Scale iPSC Cardiomyocyte Manufacturing in a Closed Process

A Polystyrene CellSTACK®-2 Chamber (Corning, cat. nr. 3269) was provided and free iPSC culture was fed around 70% confluent grown in mTeSR1 (Stem cell technologies)/Matrigel (corning) for three days. Cells were washed twice with 100 mL PBS (without Ca2+ and Mg2+, warmed to 37° C.). The cells were dissociated with 60 mL of pre-warmed Accutase per flask and incubated at 37° C. for no more than 4 minutes. The side of the flask was tapped to dislodge the cells from the surface. The cells were resuspended by pipetting up-and-down with a 50 mL stripette. The detached cells were transferred to a 500 mL conical centrifuge tube containing 100 ml mTeSR medium with 10 μM Y-27632. The flask was rinsed twice with 100 ml mTeSR containing 10 μM Y-27632 and transferred into the centrifuge tube. The tubes were centrifuged at 250 g for 10 min and resuspended (combined pellets) in a final 100 mL StemBrew (Miltenyi) containing 10 μM Y27632. The cells were counted using a Hemocytometer. The inoculum (final volume of inoculum 500 mL, final cell density in bioreactor was set to 200,000/mL) was prepared and cell suspension was added via the harvest port to the bioreactor (BioFlo Eppendorf) (containing 2.5 L Stembrew with 10 μM Y27632) by air pressure. The closed culture system (3 L) was run for 72 hours (15% DO (dissolved oxygen), 130 rpm, 37 C, pH 7.15-7.4, controlled using NaHCO₃ to avoid acidification of the medium).

‘CDM’ was prepared by mixing 0.25% albumin, 0.125% polyvinyl alcohol (Sigma-Aldrich) 1% chemically defined lipid concentrate (Gibco) 0.5% pen/strep (Gibco), 0.001% Trace-elements B (Corning), 0.01% Trace-elements C (Corning), 2 mM GlutaMAX (Gibco), 0.05 mg/ml ascorbic acid (Sigma-Aldrich), 450 microM alpha-monothioglycerol (Sigma-aldrich) in IMDM/F12 media (Gibco). ‘CDM-maturation’ was prepared by adding 1% ITS-X (Gibco), Creatine (5.7 mM), Carnitine(2 mM), Taurine(2.5 mM) and Thyroid hormone (44.5 nM) to CDM medium.

After 72 hours (day 0) the biomass (cell aggregates) was let settled for one hour. The conditioned medium (2.2 L) was pumped into the collection bottle/bag and 2.5 L CDM containing 5 μM Chir99021 to activate Wnt signaling and initiate differentiation was pumped into the bioreactor/culture vessel. Stirring was started right after the start of new medium addition. After 24 hours (day 1) the biomass was let settled for one hour. Subsequently, 2.5 L of the conditioned medium was pumped into the collection bottle/bag. Then, 2.5 L CDM supplemented with 5 μM Chir99021 was pumped into the bioreactor.

After 24 hours (day 2) the biomass was let settled for one hour. The conditioned medium (2.5 L) was pumped into the collection bottle/bag and 2.5 L CDM containing 5 μM Xav939 and 0.25 μM IWPL6 to inhibit Wnt signaling was pumped into the bioreactor.

After 24 hours (day 3) the biomass was let settled for one hour. The conditioned medium (2.5 L) was pumped into the collection bottle/bag and 2.5 L CDM containing 5 μM Xav939 and 0.25 μM IWPL6 to inhibit Wnt signaling was pumped into the bioreactor.

At day 4 the used medium was pumped into the collection bottle/bag. Subsequently, 2.5 L CDM supplemented with 0.1% ITS-X (Gibco) was pumped into the bioreactors. Stirring was started right after the start of new medium addition.

After 24 hours (day 5) the biomass was let settled for one hour. The used medium (2.5 L) was pumped into the collection bottle/bag and 2.5 L CDM supplemented with 0.1% ITS-X was pumped into the bioreactor. This step was repeated at day 6, 7, 8, 9, 10, 11, 12 and 13. Optionally, the medium was changed for CDM-maturation medium, preferably at day 7.

Harvest and Cryopreservation of iPSC-CM

The bioreactor (about 3.3 L culture medium) was stopped and the aggregates, with a typical size of 400-600 μm (e.g at day 14) were let settled for 30 min. About 2.5 L of the medium was pumped away and the remaining biomass (about 800 mL) was harvested via the harvest port (using gas pressure) into a 2 L bottle. The aggregate suspension was collected from the 2 L bottle in a collection tube. Identity of cells was subsequently measured by flow cytometric analysis. Cells were dissociated with 1× or 10× TrypLE™ Select Enzyme (Life Technologies), washed with PBS and fixed and permeabilized with Inside Fix (Miltenyi). Samples were incubated with Troponin T (TNNT2) antibodies or isotype controls (Miltenyi dilution according manufactures instructions). Samples were analyzed on a Novocyte™ Flow Cytometer (ACEA Biosciences) and compared with appropriate isotype controls (Miltenyi). Total cell counts were ˜3-5 M(illion) cells per mL and the cells positive for the marker Troponin T was >80%, indicating a conversion of iPSC to cardiomyocyte of ˜15. Aggregates were either cryopreserved as single cells after enzyme-based dissociation or as aggregates in a suitable medium containing cryoprotective agents.

### Example 2—Closed Process for Generating Endothelial Aggregates

iPSCs were inoculated in the bioreactor (Dasbox Eppendorf) at a density of 40,000/mL in 250 ml StemMACS™ iPS-Brew XF (Miltenyi) containing 10 μM Y-27632 to promote aggregate formation, pH was controlled using NaHCO3 in the range of pH 7.2 and 7.4. The stirring speed was 200 rpm (DO 15%, 37° C.). After 72 hours (day 0) the biomass (aggregates with a typical size of 50-70 μm) was let settled for one hour. Conditioned medium (200 mL) was pumped into the collection bottle/bag.

‘EDM’ was prepared by mixing 0.25% albumin, 1% chemically defined lipid concentrate (Gibco) 0.5% pen/strep (gibco), 0.001% Trace-elements B, 0.01% Trace-elements C, 2 mM GlutaMAX, 0.05 mg/ml ascorbic acid (Sigma-Aldrich), 450 microM alpha-monothioglycerol (Sigma-aldrich) in IMDM/F12 media (Gibco).

Differentiation was induced by adding 200 mL EDM containing 10 μM CHIR99021 (Axon Medchem)+31.25 ng/mL BMP4 (R&D Systems) to the bioreactor.

After 72 h of differentiation, aggregates were allowed to settle and 200 mL of Conditioned medium (200 mL) was pumped into the collection bottle/bag and 200 mL of EDM containing 62.5 ng/mL VEGF and 12.5 μM SB431542 (Tocris) was added.

After 120 h of differentiation, aggregates were allowed to settle and 200 mL of Conditioned medium (200 mL) was pumped into the collection bottle/bag and 200 mL of EDM containing 62.5 ng/mL VEGF and 12.5 μM SB431542 (Tocris) was added.

After 168 h of differentiation, aggregates were allowed to settle and 200 mL of Conditioned medium (200 mL) was pumped into the collection bottle/bag and the remaining biomass with aggregate size of 200-500 μm (about 50 mL) was harvested via the harvest port (using gas pressure) into a tube for cryopreservation.

Purities were measured by flow cytometry for the endothelial markers CD31 and CD144, typical purity was ˜60% endothelial cells and the conversation rate (yield) from iPSC to endothelial cells was ˜15. Purity could be further increased by sorting using magnetic beads such as the Miltenyi CD31+ microbead kit.

### Example 3—Closed Process for Generating Single Cell Monocytes

HiPSC aggregates of 50-70 μm were generated in a 250 mL bioreactor in mTeSR1 medium (stem cell technologies) analogous as to what is described above. To initiate differentiation, aggregates were allowed to settle for 1-hour. 200 mL of Conditioned medium (200 mL) was pumped into the collection bottle/bag.

Differentiation was induced by adding 200 mL mTeSR1 containing 62.5 ng/ml BMP4, 62.5 ng/ml VEGF, 25 ng/ml SCF was added to the bioreactor.

After 48 h of differentiation, aggregates were allowed to settle and 200 mL of Conditioned medium (200 mL) was pumped into the collection bottle/bag and 200 mL of mTeSR1 containing 50 ng/ml BMP4, 50 ng/ml VEGF, 20 ng/ml SCF was added to the bioreactor.

After 96 h of differentiation, aggregates were allowed to settle and 200 mL of Conditioned medium (200 mL) was pumped into the collection bottle/bag differentiation and differentiation was continued directly to the monocyte lineage by adding XVIVO15 (Lonza) basal medium supplemented with IL3 25 ng/ml and M-CSF 100 ng/ml to promote the formation of monocytes in the culture medium. This process was repeated at day 11, 18, 25, 32, 39 and 46. Batches of floating CD11b, CD45, and CD14 positive monocytes were harvested from the collection flask/bag at day 32, 39 and 46 and cryopreserved using processes known to the person skilled in the art. Monocytes were >90% positive for CD14, CD11b and C45 and the conversion rate (yield) was 1 stem cell to ˜12 monocytes.

### Example 4—Closed Process for Generating Single Cell HPCs and Monocytes

HiPSC aggregates were generated in a 250 mL bioreactor as described above. To initiate differentiation, aggregates were allowed to settle for 1-hour. 200 mL of Conditioned medium (200 mL) was pumped into the collection bottle/bag.

The HDM medium was prepared by mixing 0.25% albumin, 0.1% methylcellulose (Sigma-Aldrich), 0.1% polyvinyl alcohol (Sigma-Aldrich), 1×GlutaMAX, 1×ascorbic acid-2-phosphate (Sigma-Aldrich), 1% chemically defined lipid concentrate (invitrogen), 1% ITS-X, 2-mercaptoethanol (22 nM) and protein-free hybridoma mix II (4%) in IMDM/F12 media. The STAGE I supplements were: CHIR99021 (final concentration 0.5 μM; Tocris), activin A (final concentration 10 ng/ml; R&D Systems), BMP4 (final concentration 20-40 ng/ml; R&D Systems), SCF (final concentration 20 ng/ml), VEGF (final concentration 20 ng/ml) and bFGF (final concentration 5-10 ng/ml). The STAGE II supplements were: CHIR99021 (0.5 μM), activin A (10 ng/ml), BMP4 (20 ng/ml), SCF (20 ng/ml), VEGF (20 ng/ml) and bFGF (10 ng/ml). The STAGE III supplements were: CHIR99021 (3 μM), SB-431542 (3 μM; Cayman Chemical), BMP4 (20 ng/ml), SCF (20 ng/ml), VEGF (20 ng/ml) and bFGF (10 ng/ml). The STAGE IV supplements were: BMP4 (20 ng/ml), VEGF (50 ng/ml), SCF (50 ng/ml), IGFII (20 ng/ml) and bFGF (10 ng/ml).

Differentiation was induced by adding 200 mL HDM medium+stage I supplements to the bioreactor. After 24 h of differentiation, aggregates were allowed to settle and 200 mL of Conditioned medium (200 mL) was pumped into the collection bottle/bag and 200 mL of HDM containing stage II supplements was added.

After 48 h of differentiation, aggregates were allowed to settle and 200 mL of Conditioned medium (200 mL) was pumped into the collection bottle/bag and 200 mL of HDM containing stage III supplements was added.

After 72 h of differentiation, aggregates were allowed to settle and 200 mL of Conditioned medium (200 mL) was pumped into the collection bottle/bag and 200 mL of HDM containing stage III supplements was added.

After 96 h of differentiation, aggregates were allowed to settle and 200 mL of Conditioned medium (200 mL) was pumped into the collection bottle/bag and 200 mL of HDM containing stage IV supplements was added.

After 144 h of differentiation, aggregates were allowed to settle and 200 mL of Conditioned medium (200 mL) was pumped into the collection bottle/bag and 200 mL of HDM containing stage IV supplements was added.

After 192 h of differentiation, aggregates were allowed to settle and 200 mL of Conditioned medium (200 mL) was pumped into the collection bottle/bag differentiation and differentiation was continued directly to the monocyte lineage by adding XVIVO15 (lonza) basal medium supplemented with IL3 25 ng/ml and M-CSF 100 ng/ml every 3-7 days. From day 21 onwards single floating monocytes expressing >90% CD14, CD45, CD11b could be harvested from the culture medium and the conversion rate (yield) was 1 stem cell to ˜100 monocytes.

Alternatively to promote the formation of CD34, CD45 positive Hematopoietic progenitor cells (HPCs) the medium was switched at 196 h to HDM supplemented with cocktail 1 consisting of VEGF (50 ng ml-1), SCF (100 ng ml-1), bFGF (10 ng ml-1), FLT3L (10 ng ml-1) and IL3 (10 ng ml-1) or HDM supplemented with cocktail 2 consisting of TPO (10-25 ng/ml), SCF (10-25 ng/ml), Flt3L (10-25 ng/ml), IL-3 (2-10 ng/ml), IL-6 (2-10 ng/ml), SRI (0.75 mM), OSM (2-10 ng/ml), and EPO (2 U/ml).

For both the HPC and the monocyte process the medium was changed every 3-7 days up to day 45, using the methods described above. The single cells were isolated from the collection bottle for further processing and aggregates were allowed to settle for further culturing.

### Example 5—Closed Process for Generating Cortical Neurons from hiPSC

HiPSC aggregates were generated in a 250 mL bioreactor in mTeSR1 medium (stem cell technologies) as described above. To initiate differentiation, aggregates were allowed to settle for 1-hour. 200 mL of Conditioned medium (200 mL) was pumped into the collection bottle/bag.

Differentiation was induced by adding 200 mL mTeSR1 containing 12.5 μM activin/TGF-b inhibitor SB431542 (R&D Systems) and 1.25 μM BMP inhibitor LDN193189 (Stemgent) to the bioreactor.

The NDM medium I was prepared by mixing 15% KSR (Invitrogen), KO DMEM (Invitrogen), 2 mM L-glutamine (Gibco), 1% non-essential amino acids (NEAA) (Gibco), 1% penicillin-streptomycin (Gibco), and 50 μM β-mercaptoethanol (Gibco).

The NDM medium II was prepared by mixing DMEM/F12 (Invitrogen), 1% N2 supplement (Gibco), 2% B27 supplement without vitamin A (Life Technologies) 1% Glutamax (Gibco), 1% NEAA (Gibco), 1% penicillin-streptomycin (Gibco).

After 24 h, 48 h of differentiation, aggregates were allowed to settle and 200 mL of Conditioned medium (200 mL) was pumped into the collection bottle/bag and 200 mL of NDM medium I containing 2 μM XAV939, 10 μM SB431542 (R&D Systems) and 1.25 μM BMP inhibitor LDN193189 (Stemgent) was added to the bioreactor.

After 72 h of differentiation, aggregates were allowed to settle and 200 mL of Conditioned medium (200 mL) was pumped into the collection bottle/bag and 200 mL of NDM medium I, 10 μM SB431542 (R&D Systems) and 1.25 μM BMP inhibitor LDN193189 (Stemgent) was added to the bioreactor.

At day 5 of differentiation, aggregates were allowed to settle and 200 mL of Conditioned medium (200 mL) was pumped into the collection bottle/bag and 200 mL of NDM medium I/II (68.75%/31.25%) containing 10 μM SB431542 (R&D Systems) and 1.25 μM BMP inhibitor LDN193189 (Stemgent) was added to the bioreactor.

At day 6 of differentiation, aggregates were allowed to settle and 200 mL of Conditioned medium (200 mL) was pumped into the collection bottle/bag and 200 mL of NDM medium I/II (43.75%/56.25%) containing 10 μM SB431542 (R&D Systems) and 1.25 μM BMP inhibitor LDN193189 (Stemgent) was added to the bioreactor.

At day 8 of differentiation, aggregates were allowed to settle and 200 mL of Conditioned medium (200 mL) was pumped into the collection bottle/bag and 200 mL of NDM medium I/II (18.75%/81.25%) was added to the bioreactor.

At day 10, 13 ad 17 of differentiation, aggregates were allowed to settle and 200 mL of Conditioned medium (200 mL) was pumped into the collection bottle/bag and 200 mL of NDM medium II was added to the bioreactor.

At day 20, 23, 27, 30 of differentiation, aggregates were allowed to settle and 200 mL of Conditioned medium (200 mL) was pumped into the collection bottle/bag and 200 mL of NDM medium II containing 10 ng/ml brain-derived neurotrophic factor (BDNF) and 10 ng/ml glial cell-derived neurotrophic factor (GDNF) (both from R&D systems) was added to the bioreactor.

### Example 6

iPSC aggregates were differentiated during 6 days to hemogenic endothelium with two media refresh strategies wherein either part of the medium (e.g. 70 vol. %, 80 vol. % and/or 90 vol. %) or all of the medium was refreshed at day 2, 3, 4 and 6. Cell counts were measured at day 6. The strategy wherein part of the media was refreshed on the different days showed increased the total cell number, in this experiment by more than 117% (more than 2×), and the cell type of interest (CD34+, CD73−) was increased by at least 10%. Without being bound by theory, these results suggest that cultures are performing better with a strategy that includes partial replacement of culture medium versus (substantially) full replacement, which could be due to reduced stress, reduced loss of cells due to manipulation, or beneficial cytokines secreted by the cells.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.//

1. Method for the in vitro manufacture of a preselected cell type differentiated from a pluripotent stem cell, preferably in a closed culture system, wherein the method comprises the steps of:
a) providing pluripotent stem cells and a culture medium;
b) introducing the pluripotent stem cells and the culture medium into a culture vessel, preferably wherein the culture vessel is part of a closed culture system, wherein the culture medium is
i) a culture medium for proliferation of the pluripotent stem cells; or
ii) a culture medium for inducing differentiation of the pluripotent stem cells towards the preselected cell type;
c) mixing the culture medium in the culture vessel thereby allowing the cells to grow in the form of cell aggregates and preventing settling of the cell aggregates;
d) discontinuing the mixing of the culture medium in the culture vessel thereby allowing the cell aggregates to settle;
e) collecting part of the culture medium in the culture vessel;
f) optionally, in case in step b) a culture medium for proliferation of the pluripotent stem cells was used, introducing a further culture medium for proliferation of the pluripotent stem cells in the culture vessel and repeating step c)-e);
g) introducing a subsequent culture medium into the culture vessel, wherein the culture medium is a culture medium for inducing differentiation of the cells towards the preselected cell type;
h) mixing the culture medium in the culture vessel thereby allowing the cells to grow in the form of cell aggregates and preventing settling of the cell aggregates;
i) discontinuing the mixing of the culture medium in the culture vessel thereby allowing the cell aggregates to settle;
j) collecting part of the culture medium in the culture vessel and repeating steps g)-i) for a subsequent culture medium, or collecting the culture medium in the culture vessel, collecting the cell aggregates in the culture vessel, or collecting both.

2. Method according to claim 1, wherein steps g)-i) is repeated once or more than once, using one or more subsequent culture media.

3. Method according to any one of the previous claims, wherein in step b) the pluripotent stem cells are introduced in the form of a single cell suspension or wherein in step b) the pluripotent stem cells are introduced in the form of cell aggregates.

4. Method according to any one of the previous claims, wherein in step b) the amount of pluripotent stem cells in the culture medium is between 1×10⁴-1×10⁶ pluripotent stem cells per ml culture medium.

5. Method according to any one of the previous claims, wherein, when the cells in step b) are introduced in the form of cell aggregates, the cell aggregates have a size of between 10-150 micrometer, preferably between 25-140 micrometer, preferably selected from the group consisting of between 20-80 micrometer, 30-60 micrometer, 90-140 micrometer, and 100-120 micrometer.

6. Method according to any one of the previous claims wherein the amount of preselected cell type manufactured is at least 10 times the amount of pluripotent stem cells introduced in step b) or the amount of step cells induced to differentiate towards the preselected cell type in step b) or in step g), preferably at least 15 times, at least 20 times, or at least 25 times, preferably between 10-100 times, between 15-80 times or between 20-75 times.

7. Method according to any one of the previous claims wherein the cell aggregates collected in step j) have a size of less than 1000 micrometer, preferably have a size of between 10-1000 micrometer, 20-750 micrometer or 50-500 micrometer.

8. Method according to any one of the previous claims wherein the volume of the culture medium in the culture vessel is at least 1 liter, preferably at least 2 liter, 3 liter, 4 liter, 5 liter, 6 liter, 7.5 liter, or 10 liter, preferably wherein the culture medium in the culture vessel is between 1 liter and 100 liter, preferably between 5 liter and 50 liter.

9. Method according to any one of the previous claims wherein at least 1×10⁶ cells/ml culture medium is manufactured, preferably at least 1.5×10⁶ cells/ml, 2.0×10⁶ cells/ml, 3.0×10⁶ cells/ml, 4.0×10⁶ cells/ml, 5.0×10⁶ cells/ml, 8.0×10⁶ cells/ml, 12.0×10⁶ cells/ml or 200×10⁶ cells/ml.

10. Method according to any one of the previous claims wherein at least 1×10⁹ preselected cells are manufactured, preferably at least 10×10⁹, 25×10⁹, 100×10⁹, 200×10⁹, or 500×10⁹ preselected cells.

11. Method according to any one of the previous claims wherein optional step f) is omitted and/or wherein step g)-i) are repeated at least once, preferably at least 2, 3, 4, 5, 6, 7, 8, 9, 10, 15, 20, 25 times.

12. Method according to any one of the previous claims wherein step c) or h) is, each independently, for at least 12 hours, 1, 2, 3, 4, 5, 6, or 7 days, preferably no more than 10, preferably no more than 7 days and/or wherein step h) is for at least 12 hours, 1, 2, 3, 4, 5, 6, or 7 days.

13. Method according to any one of the previous claims wherein the method is performed over a period of time of at least 5, 6, 7, 10, 11, 12, 13, 14, 15, 16, 17, 18, 19, 20 days, preferably between 7 and 90 days, 10-60 days, 15-40 days, or 15-30 days.

14. Method according to any of the preceding claims, wherein the compositions of the different culture media for proliferation of the pluripotent stem cells are the same or different and/or wherein the compositions of the different culture media for differentiation of the cells towards the preselected cell type are the same or different.

15. Method according to any of the preceding claims, wherein in step e) and/or j) at most 95 vol. %, 90 vol. %, 85 vol. %, or 80 vol. % of the culture medium in the culture vessel is collected and/or wherein in step e) and/or j) at least 50 vol. %, 60 vol. % or 70 vol. % of the culture medium in the culture vessel is collected.

16. Method according to any of the preceding claims, wherein the preselected cell type is a cardiovascular cell, a cardiomyocyte, an endothelial cell, a cell of the hematopoietic linage, a hematopoietic progenitor cell, a cell differentiated from a hematopoietic progenitor cell, a monocyte, a macrophage, a T-cell, a B-cell, a NK-cell, a dendritic cell, a neuronal cell, a retinal cell, a lung cell, a liver cell, or a pancreatic cell.

17. Method according to any of the preceding claims, wherein the part of the culture medium that is collected or the culture medium that is collected comprises single cells and/or non-aggregated cells, preferably wherein these single cells or non-aggregated cells are selected from the group consisting of a cell of the hematopoietic linage, a hematopoietic progenitor cell, a cell differentiated from a hematopoietic progenitor cell, a monocyte, a macrophage, a T-cell, a B-cell, a NK-cell, or a dendritic cell.

18. Method according to any of the preceding claims, wherein the culture medium provided in step b) or, optionally, step g) comprises a Rho-associated protein kinase inhibitor.

19. Method according to any of the preceding claims, wherein the culture medium provided in step b) and/or g), preferably step g) comprises polyvinyl alcohol.

20. Method according to any of the preceding claims, wherein the culture medium for inducing differentiation of the cells towards the preselected cell type comprises one or more compounds that induce differentiation of the cells towards the preselected cell type, preferably wherein the one or more compounds are selected from the group consisting of Wnt-pathway activators, Wnt-pathway inhibitors, Activin-pathway activators, TGFβ-pathway activators, BMP-pathway activators, Activin-pathway inhibitors, TGFβ-pathway inhibitors, BMP-pathway inhibitors, and VEGF-pathway activators.

21. Method according to any of the previous claims, wherein the one or more compounds that induce the differentiation of the (pluripotent stem) cells towards the preselected cell type is/are selected from the group consisting of compounds that induce the differentiation of the cells to a cardiovascular cell, a cardiomyocyte, an endothelial cell, a cell of the hematopoietic linage, a hematopoietic progenitor cell, a cell differentiated from a hematopoietic progenitor cell, a monocyte, a macrophage, a T-cell, a B-cell, a NK-cell, a dendritic cell, a neuronal cell, a retinal cell, a lung cell, a liver cell, or a pancreatic cell.

22. Method according to any of the preceding claims, wherein the culture media for inducing differentiation of the (pluripotent stem) cells towards the preselected cell type comprises a thyroid hormone and/or thyroid hormone analog.

23. Method according to any of the preceding claims, wherein the pluripotent stem cells is an induced pluripotent stem cell, a human pluripotent stem cell, or a human induced pluripotent stem cell.

24. Method according to any of the preceding claims wherein the preselected cell type cell is obtained from the cell aggregate or from the part of the culture medium that is collected or from the culture medium that is collected.

25. Method according to any of the preceding claims wherein the in vitro manufactured preselected cell type is for use in cell therapy.

26. Use of a closed culture system in the in vitro manufacture of a preselected cell type differentiated from a pluripotent stem cell, preferably according to a method as defined in any one of the previous claims.

27. A, preferably closed, culture system comprising at least 1×10⁹ preselected cells, preferably at least 10×10⁹, 25×10⁹, 100×10⁹, 200×10⁹, or 500×10⁹ preselected cells and/or comprising at least 1 liter, preferably at least 2 liter, 3 liter, 4 liter, 5 liter, 6 liter, 7.5 liter, or 10 liter culture medium, preferably between 1 liter and 100 liter, preferably between 5 liter and 50 liter culture medium comprising at least 1×10⁶ cells/ml culture medium, preferably at least 1.5×10⁶ cells/ml, 2.0×10⁶ cells/ml, 3.0×10⁶ cells/ml, or 5.0×10⁶ cells/ml.

28. Pharmaceutical composition for use in cell therapy wherein the cell therapy comprises the step of providing a preselected cell type to a subject in need thereof and wherein the preselected cell type has been manufactured with a method as defined in any of the previous method claims or wherein the cell therapy comprises the step of manufacture of a preselected cell type with a method as defined in any of the previous method claims and providing the preselected cell type to a subject in need thereof.
